(19) United States
(12) Patent Application Publication
(10) Pub. No.: US 2007/0219254 A1
(43) Pub. Date: Sep. 20, 2007

(54) Therapeutic Compounds: Pyridine N-Oxide Scaffold

(76) Inventors: Ziping Liu, Montreal (CA); Christopher Walpole, Montreal (CA); Zhong-Yong Wei, Montreal (CA); Hua Yang, Montreal (CA)

Correspondence Address: WHITE & CASE LLP;PATENT DEPARTMENT, 1155 AVENUE OF THE AMERICAS, NEW YORK, NY, 10036, US

(21) Appl. No.: 11/569,326

(22) PCT Filed: May 20, 2005
(86) PCT No.: PCT/SE05/00752

§ 371 Date: Nov. 17, 2006

(30) Foreign Application Priority Data
May 25, 2004 (SE) 0401343-9

(51) Int. Cl. A61K 31/44; A61K 31/4433; A61P 25/00; C07D 213/89; C07D 405/12

(52) U.S. Cl. 514/352; 546/310

(57) ABSTRACT

Compounds of formula I or pharmaceutically acceptable salts thereof: wherein R¹, R², R³, R⁴, m and n are as defined in the specification as well as salts and pharmaceutical compositions including the compounds are prepared. They are useful in therapy, in particular in the management of pain.

### BACKGROUND OF THE INVENTION

1. Field of the Invention

The invention is related to therapeutic compounds, pharmaceutical compositions containing these compounds, manufacturing processes thereof and uses thereof. Particularly, the present invention is related to compounds that may be effective in treating pain, cancer, multiple sclerosis, Parkinson's disease, Huntington's chorea, Alzheimer's disease, anxiety disorders, gastrointestinal disorders and/or cardiavascular disorders.

2. Discussion of Relevant Technology

Pain management has been an important field of study for many years. It has been well known that cannabinoid receptor (e.g., CB₁ receptor, CB₂ receptor) ligands including agonists, antagonists and inverse agonists produce relief of pain in a variety of animal models by interacting with CB₁ and/or CB₂ receptors. Generally, CB₁ receptors are located predominately in the central nervous system, whereas CB₂ receptors are located primarily in the periphery and are primarily restricted to the cells and tissues derived from the immune system.

While CB₁ receptor agonists, such as Δ⁹-tetrahydrocannabinol (Δ⁹-THC) and anadamide, are useful in anti-nociception models in animals, they tend to exert undesired CNS side effects, e.g., psychoactive side effects, the abuse potential, drug dependence and tolerance, etc. These undesired side effects are known to be mediated by the CB₁ receptors located in CNS. There are lines of evidence, however, suggesting that CB 1 agonists acting at peripheral sites or with limited CNS exposure can manage pain in humans or animals with much improved overall in vivo profile.

Therefore, there is a need for new CB₁ receptor ligands such as agonists that may be useful in managing pain or treating other related symptoms or diseases with reduced or minimal undesirable CNS side effects.

### DESCRIPTION OF THE EMBODIMENTS

The present invention provides CB₁ receptor ligands that may be useful in treating pain and/or other related symptoms or diseases.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-i-butyl, 2-methyl-3-butyl, 2,2-dimethyl-1-propyl, 2-methyl-1-pentyl, 3-methyl-i-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethyihexenyl, 2-propyl-2-butenyl, 4-(2-methyl-3-butene)-pentenyl. An alkenyl can be unsubstituted or substituted with one or two suitable substituents.

The term “alkynyl” used alone or as suffix or prefix, refers to a monovalent straight or branched chain hydrocarbon radical having at least one carbon-carbon triple bond and comprising at least 2 up to about 12 carbon atoms. The triple bond of an alkynyl group can be unconjugated or conjugated to another unsaturated group. Suitable alkynyl groups include, but are not limited to, C₂₋₆alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl can be unsubstituted or substituted with one or two suitable substituents.

The term “cycloalkyl,” used alone or as suffix or prefix, refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character.

The term “heterocylcoalkyl” used alone or as a suffix or prefix, refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amine” or “amino” used alone or as a suffix or prefix, refers to —NH₂.

Halogen includes fluorine, chlorine, bromine and iodine.

“Halogenated,” used as a prefix of a group, means one or more hydrogens on the group is replaced with one or more halogens.

“RT”, “r.t.” or “rt” means room temperature.

“mCPBA” refers to meta-Chloroperbenzoic acid.

One aspect of the invention is a compound of formula I, a pharmaceutically acceptable salt thereof, diastereomers, enantiomers, or mixtures thereof: 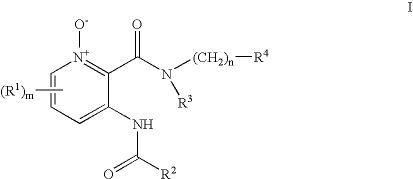
wherein:

m is selected from 0, 1 and 2;

n is selected from 0, 1, 2, 3, 4 and 5;

R¹ is independently selected from halogen, cyano, amino, nitro, C₁₋₆alkylamino, diC₁₋₆alkylamino, acetylamino, hydroxyl, C₁₋₆alkoxy, C₁₋₆alkyl, halogenated C₁₋₆alkoxy, C₁₋₆alkenyl, and halogenated C₁₋₆alkyl;

R² is selected from C₆₋₁₀aryl and C₂₋₁₀heterocyclyl; wherein said C₆₋₁₀aryl and C₂₋₁₀heterocyclyl used in defining R² is optionally substituted by one or more groups selected from halogen, halogenated C₁₋₆alkyl, C₁₋₆alkyl, cyano, nitro, C₁₋₆alkoxy, halogenated C₁₋₆atkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁aryl, and C₆₋₁₀aryl-C₁₋₆alkyl; and

R³ is selected from hydrogen and C₁₋₆alkyl; R⁴ is selected from C₁₋₆alkyl, C₃₋₇cycloalkyl, C₄₋₇cycloatkenyl, C₆₋₁₀aryl, C₂₋₆heterocyclyl-amino, C₂₋₆heterocyclyloxy-amino and C₂₋₆heterocyclyl; wherein said C₁₋₆alkyl, C₃₋₇cycloalkyl, C₄₋₇cycloalkenyl, C₆₋₁₀aryl, C₂₋₆heterocyclyi-amino, C₂₋₆heterocyclyloxy-amino and C₂₋₆heterocyclyl used in defining R⁴ is optionally substituted by one or more groups selected from halogen, halogenated C₁₋₆atkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆allylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl; or 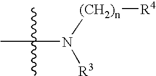
is C₂₋₁₀heterocyclyl, which is optionally substituted by one or more groups selected from halogen, halogen substituted C₁₋₆alkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C1-6alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl.

In another embodiment, the compounds of the present invention are those of formula I,

wherein

m is selected from 0, 1 and 2;

n is selected from 0, 1, 2, 3 and 4;

R¹ is independently selected from halogen, cyano, amino, nitro, acetylamino, hydroxyl, C1-3alkoxy, C₁₋₃alkyl, halogenated C₁₋₃alkoxy, and halogenated C₁₋₃alkyl;

R² is selected from C₆₋₁₀aryl and C₂₋₁₀heterocyclyl, wherein said C₆₋₁₀aryl and C₂₋₁₀heterocyclyl used in defining R² is optionally substituted by one or more groups selected from halogen, halogenated C₁₋₃alkyl, C₁₋₃alkyl, nitro, C₁₋₃alkoxy, halogenated C₁₋₃alkoxy, hydroxy, hydroxy-C₁₋₃alkyl, amino, C₁₋₃alkoxy-C₁₋₃alkyl, C₂₋₅heterocyclyl-C₁₋₃alkyl, C₁₋₆alkoxycarbonyl, C₁₋₃alkylamino, diC₁₋₃alkyl-amino, amino-C₁₋₃alkyl; and

R³ is selected from hydrogen and C₁₋₆ alkyl; R⁴ is selected from C₁₋₆alkyl, C₃₋₇cycloalkyl, C₂₋₆heterocyclyl-amino, C₂₋₆heterocyclyloxy-amino, and C₂₋₆heterocyclyl; wherein said C₁₋₆alkyl, C₃₋₇cycloalkyl, C₂₋₆heterocyclyl-amino, C₂₋₆heterocyclyloxy-amino, and C₂₋₆heterocyclyl used in defining R⁴ is optionally substituted by one or more groups selected from halogen, halogenated C1-3alkyl, C₁₋₃alkyl, nitro, C₁₋₃alkoxy, halogenated C₁₋₃alkoxy, hydroxy, hydroxy-C₁₋₃alkyl, amino, C₁₋₃alkoxy-C₁₋₃alkyl, C₁₋₆alkoxycarbonyl, C₁₋₃alkylamino, diC₁₋₃alkyl-amino, and amino-C₁₋₃alkyl; or 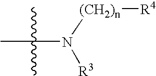
is selected from azepanyl, pyrrolyl, pyrrolinyl, pyrrolidinyl, imidazolyl, imidazolidinyl, pyrazolyl, pyrazolinyl, pyrazolidinyl, isoxazolidinyl, triazolyl, morpholinyl, piperidinyl, thiomorpholinyl, pyridazinyl, piperazinyl, triazinyl or 1,4-dioxa-8-azaspiro[4.5]decan-8-yl; wherein said azepanyl, pyrrolyl, pyrrolinyl, pyrrolidinyl, imidazolyl, imidazolidinyl, pyrazolyl, pyrazolinyl, pyrazolidinyl, isoxazolidinyl, trazolyl, morpholinyl, piperidinyl, thiomorpholinyl, piperazinyl, triazinyl and 1,4-dioxa-8-azaspiro[4.5]decan-8-yl are optionally substituted by one or more groups selected from halogen, halogenated C₁₋₃alkyl, C₁₋₃alkyl, nitro, C₁₋₃alkoxy, halogenated C₁₋₃alkoxy, hydroxy, hydroxy-C₁₋₃alkyl, amino, C₁₋₃alkoxy-C₁₋₃alkyl, C₁₋₆alkoxycarbonyl, C₁₋₃alkylamino, diC₁₋₃alkyl-amino, and amino-C₁₋₃alkyl.

In a further embodiment, the compounds of the present invention are those of formula I, wherein

m is selected from 0 and 1;

n is selected from 0, 1, 2, 3 and 4;

R¹ is independently selected from halogen, amino, nitro, acetylamino, hydroxyl, C₁₋₃alkoxy, C₁₋₃alkyl, halogenated C₁₋₃alkoxy, and halogenated C₁₋₃alkyl;

R² is selected from phenyl, naphthyl, pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl, indolyl, indolinyl, quinolinyl, tetrahydroquinolinyl, isoquinolinyl, tetrahydroisoquinolinyl, 1,4-benzodioxanyl, coumarin, dihydrocoumarinyl, 2,3-dihydrobenzofuranyl, 1,2-benzisoxazolyl, 1,3-benzodioxolyl, 2,3-dihydro-1,4-benzodioxinyl, 3,4-dihydro-2H-1,5-benzodioxepinyl, 4H-1,3-benzodioxinyl, benzofuranyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl that are optionally substituted by one or more groups selected from halogen, hydroxy, methyl, methoxy, amino, trifluoromethyl, trifluoromethoxy, methoxymethyl, 1H-1,2,3-triazolylmethyl and 1H-pyrazolylmethyl;

R³ is selected from hydrogen and C₁₋₆ alkyl; and

R⁴ is selected from 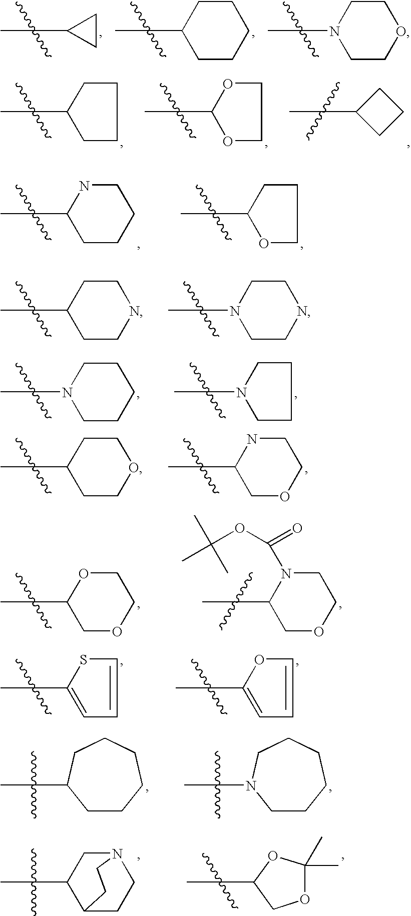
pyrrolidin-1-amino, piperidin-1-amino, O-cyclohexylhydroxyamino, O-cyclopentylhydroxyamino, O-cyclobutylhydroxyamino, O-cyclopropylhydroxyamino, and C₁₋₃alkyl that are optionally substituted by one or more groups selected from halogen, amino, aminomethyl, 2-aminoethyl, hydroxy, hydroxylmethyl, methyl and ethyl.

Particularly, R² is selected from

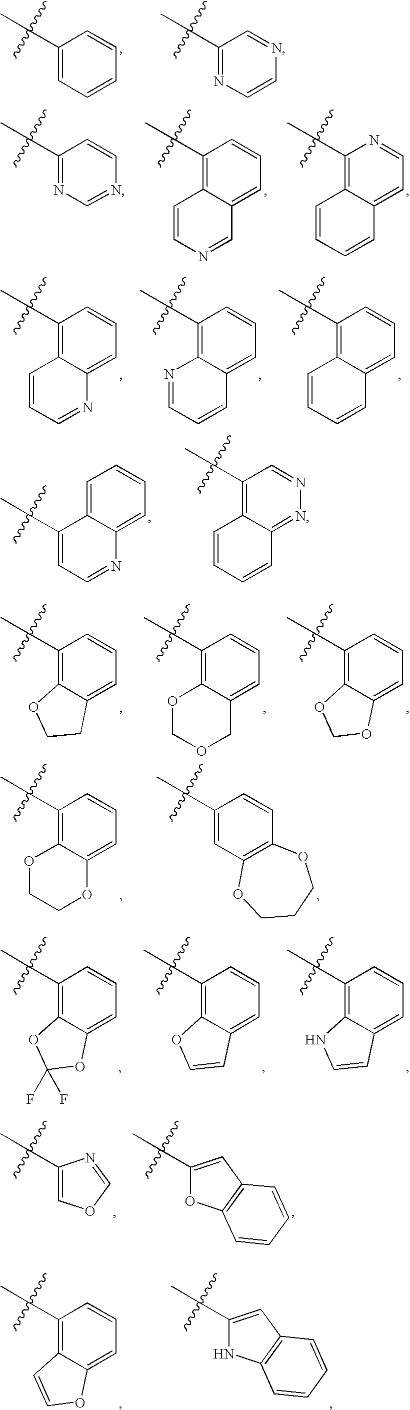

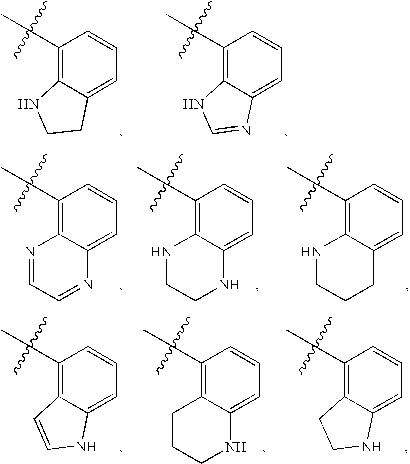

that are optionally substituted with one or more groups selected from halogen, methyl, methoxy, hydroxyl, methoxymethyl, 1H-1,2,3-triazolylmethyl and 1H-1,2-diazolylmethyl.

In an even further embodiment, the compounds of the present invention are those of formula I and pharmaceutically acceptable salts thereof,

wherein m is 1;

n is selected from 0, 1, 2, and 3;

R¹ is independently selected from halogen, amino, nitro, acetylamino, hydroxyl, C₁₋₃alkoxy, C₁₋₃alkyl, halogenated C₁₋₃alkoxy, and halogenated C₁₋₃alkyl;

R² is selected from phenyl, naphthyl, pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl, indolyl, indolinyl, quinolinyl, tetrahydroquinolinyl, isoquinolinyl, tetrahydroisoquinolinyl, 1,4-benzodioxanyl, coumarin, dihydrocoumarinyl, 2,3-dihydrobenzofuranyl, 1,2-benzisoxazolyl, 1,3-benzodioxolyl, 2,3-dihydro-1,4-benzodioxinyl, 3,4-dihydro-2H-1,5-benzodioxepinyl, 4H-1,3-benzodioxinyl, benzofuranyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl that are optionally substituted by one or more groups selected from halogen, hydroxy, methyl, methoxy, amino, trifluoromethyl, trifluoromethoxy, methoxymethyl, 1H-1,2,3-triazolylmethyl, 1H-pyrazolylmethyl; and 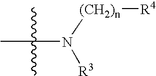
is selected from azetidinyl, azepanyl, isoxazolidinyl, morpholinyl, piperazinyl, piperidinyl, pyrrolidinyl, and 1,4-dioxa-8-azaspiro[4.5]decan-8-yl that were optionally substituted with one or more groups selected from halogen, cyano, nitro, methyl, ethyl, hydroxy, hydroxy-methyl, hydroxy-ethyl, amino-methyl, amino-ethyl, methoxy-methyl, methoxy-phenyl, ethoxycarbonyl, tert-butoxycarbonyl, diphenyl-methyl, morpholinyl-eth-2-yl, piperidinyl-methyl and pyridinyl.

Particularly, 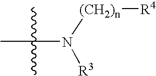
is selected from

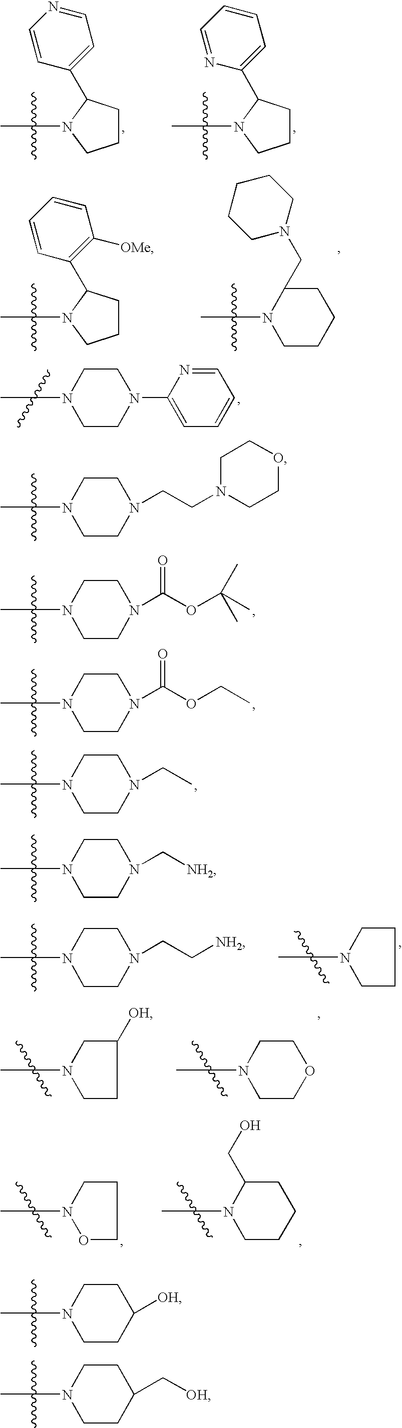

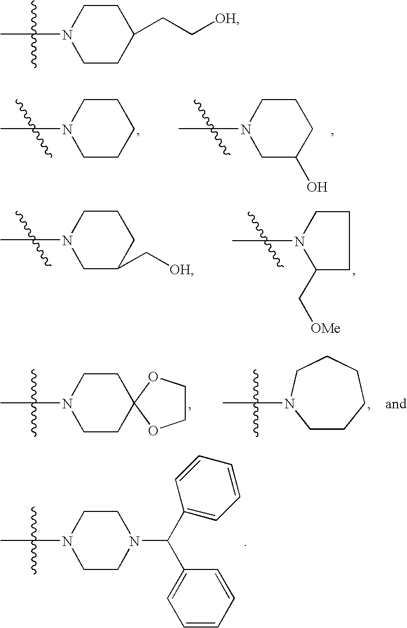

More particularly, R² is selected from

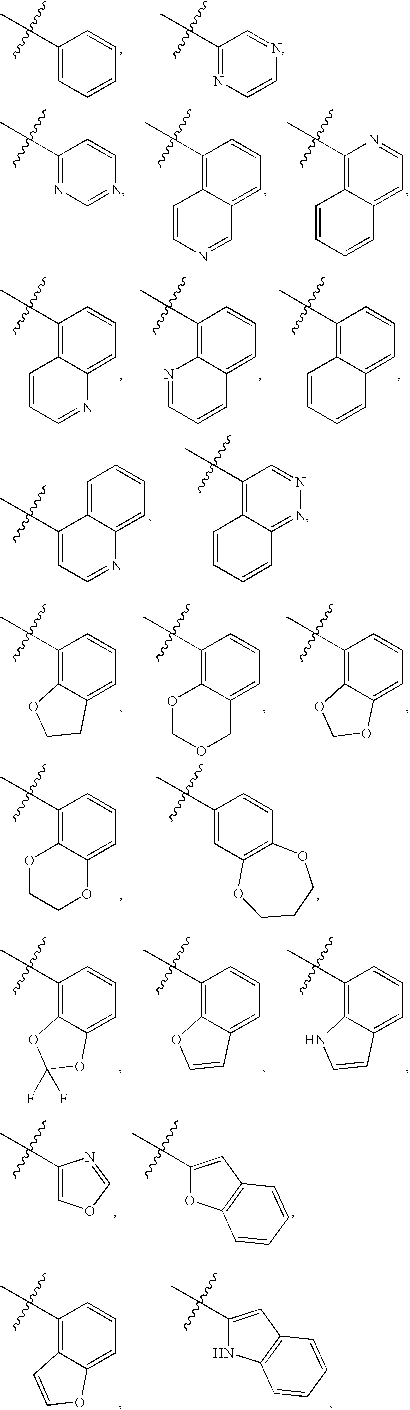

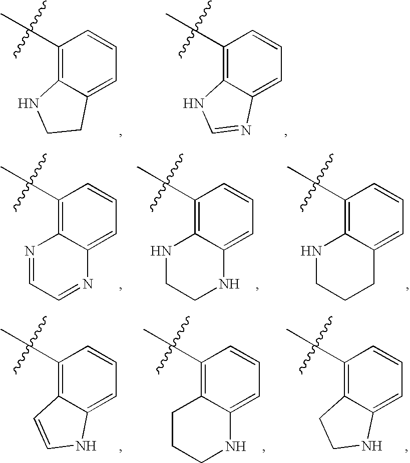

that are optionally substituted with one or more groups selected from halogen, methyl, methoxy, hydroxyl, methoxymethyl, 1H-1,2,3-triazolylmethyl and 1H-pyrazolylmethyl.

In a more particularly embodiment, R² is selected from 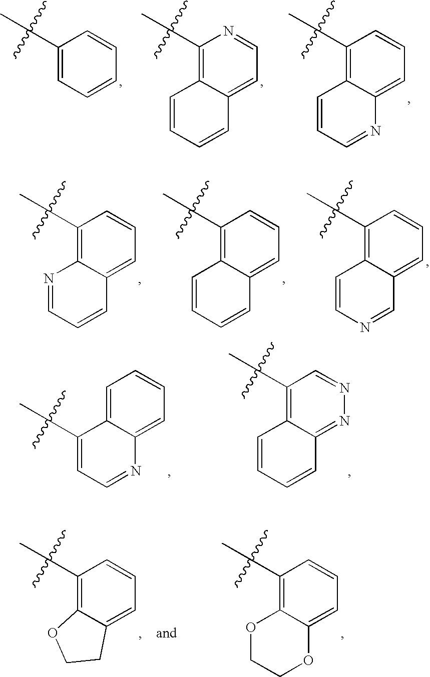
optionally substituted with one or more groups selected from halogen, methyl, methoxy, hydroxyl, methoxymethyl, 1H-1,2,3-triazolylmethyl and 1H-pyrazolylmethyl.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the formula I.

It will also be understood that certain compounds of the present invention may exist in solvated, for example hydrated, as well as unsolvated forms. It will further be understood that the present invention encompasses all such solvated forms of the compounds of the formula I.

Within the scope of the invention are also salts of the compounds of the formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as modulators or ligands such as agonists, partial agonists, inverse agonist or antagonists of CB₁ receptors. More particularly, the compounds of the invention exhibit activity as agonist of the CB, receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of CB₁ receptors is present or implicated. Furthermore, the compounds of the invention may be used to treat cancer, multiple sclerosis, Parkinson's disease, Huntington's chorea, Alzheimer's disease, anxiety disorders, gastrointestinal disorders and cardiovascular disorders.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of cannabinoid receptors is present or implicated in that paradigm. This may involve the use of isotopically labelled versions of the compounds of the invention in diagnostic techniques and imaging applications such as positron emission tomography (PET).

Compounds of the invention are useful for the treatment of diarrhoea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

In another aspect of the invention is the use of a compound according to formula I for inhibition of transient lower esophageal sphincter relaxations (TLESRs) and thus for treatment or prevention of gastroesophageal reflux disorder (GERD). The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. In further embodiments, the compounds according to the present invention are useful for the prevention of reflux, treatment or prevention of regurgitation, treatment or prevention of asthma, treatment or prevention of laryngitis, treatment or prevention of lung disease and for the management of failure to thrive.

A further aspect of the invention is the use of a compound according to formula I for the manufacture of a medicament for the inhibition of transient lower esophageal sphincter relaxations, for the treatment or prevention of GERD, for the prevention of reflux, for the treatment or prevention of regurgitation, treatment or prevention of asthma, treatment or prevention of laryngitis, treatment or prevention of lung disease and for the management of failure to thrive.

An even further aspect of the invention is the use of a compound according to formula I for the manufacture of a medicament for the treatment or prevention of functional gastrointestinal disorders, such as functional dyspepsia (FD). Yet another aspect of the invention is the use of a compound according to formula I for the manufacture of a medicament for the treatment or prevention of irritable bowel syndrome (IBS), such as constipation predominant IBS, diarrhea predominant IBS or alternating bowel movement predominant IBS. Exemplary irritable bowel syndrome (IBS) and functional gastrointestinal disorders, such as functional dyspepsia, are illustrated in Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders: Diagnosis, Pathophysiology and Treatment. 2 ed. McLean, V A: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl.2), II1-II81.9-1-1999.

Also within the scope of the invention is the use of any of the compounds according to the formula I above, for the manufacture of a medicament for the treatment of any of the conditions discussed above.

A further aspect of the invention is a method for the treatment of a subject suffering from any of the conditions discussed above, whereby an effective amount of a compound according to the formula I above, is administered to a patient in need of such treatment.

Thus, the invention provides a compound of formula I, or pharmaceutically acceptable salt or solvate thereof, as hereinbefore defined for use in therapy.

In a further aspect, the present invention provides the use of a compound of formula I, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (per cent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of formula I as defined above for the manufacture of a medicament.

Also within the scope of the invention is the use of any compound of formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the formula I above, is administered to a patient in need of such therapy.

Additionally, there is provided a pharmaceutical composition comprising a compound of Formula I, or a pharmaceutically acceptable salt thereof, in association with a pharmaceutically acceptable carrier.

Particularly, there is provided a pharmaceutical composition comprising a compound of Formula I, or a pharmaceutically acceptable salt thereof, in association with a pharmaceutically acceptable carrier for therapy, more particularly for therapy of pain.

Further, there is provided a pharmaceutical composition comprising a compound of Formula I, or a pharmaceutically acceptable salt thereof, in association with a pharmaceutically acceptable carrier use in any of the conditions discussed above.

Another aspect of the invention is a method of preparing the compounds of the present invention.

In one embodiment, the method of the invention is a method for preparing a compound of formula I, 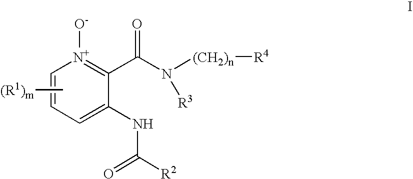
comprising the step of reacting a compound of formula II, 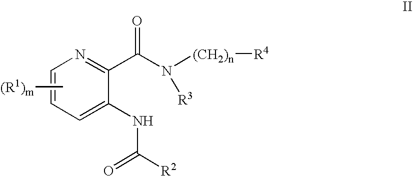
with mCPBA, in the presence of a solvent such as CH₂Cl₂,
wherein:

m is selected from 0, 1 and 2;

n is selected from 0, 1, 2, 3, 4 and 5;

R¹ is independently selected from halogen, cyano, amino, nitro, C₁₋₆alkylamino, diC₁₋₆alkylamino, acetylamino, hydroxyl, C₁₋₆alkoxy, C₁₋₆alkyl, halogenated C₁₋₆alkoxy, C₁₋₆alkenyl, and halogenated C₁₋₆atkyl;

R² is selected from C₆₋₁₀aryl and C₂₋₁₀heterocyclyl; wherein said C₆₋₁₀aryl and C₂₋₁₀heterocyclyl used in defining R² is optionally substituted by one or more groups selected from halogen, halogenated C₁₋₆alkyl, C₁₋₆alkyl, cyano, nitro, C₁₋₆alkoxy, halogenated C₁₋₆atkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₂₋₅heterocyclyl-C₁₋₃alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl; and

R³ is selected from hydrogen and C₁₋₆alkyl; R⁴ is selected from C₁₋₆alkyl, C₃₋₇cycloalkyl, C₄₋₇cycloalkenyl, C₆₋₁₀aryl, C₂₋₆heterocyclyl-amino, C₂₋₆heterocyclyloxy-amino, and C₂₋₆heterocyclyl; wherein said C₁₋₆alkyl, C₃₋₇cycloalkyl, C₄₋₇cycloalkenyl, C₆₋₁₀aryl, C₂₋₆heterocyclyl-amino, C₂₋₆heterocyclyloxy-amino, and C₂₋₆heterocyclyl used in defining R⁴ is optionally substituted by one or more groups selected from halogen, halogenated C₁₋₆alkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl; or 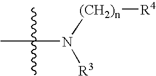
is selected from a C₂₋₁₀heterocyclyl, which is optionally substituted by one or more groups selected from halogen, halogen substituted C₁₋₆alkyl, C₁₋₆alkyl, cyano, nitro, C₁₋₆alkoxy, halogenated C₁₋₆alkoxy, hydroxy, hydroxy-C1-6alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl.

Compounds of the present invention may be prepared according to, but not limited to, the synthetic routes as depicted in Schemes 1 and 2. 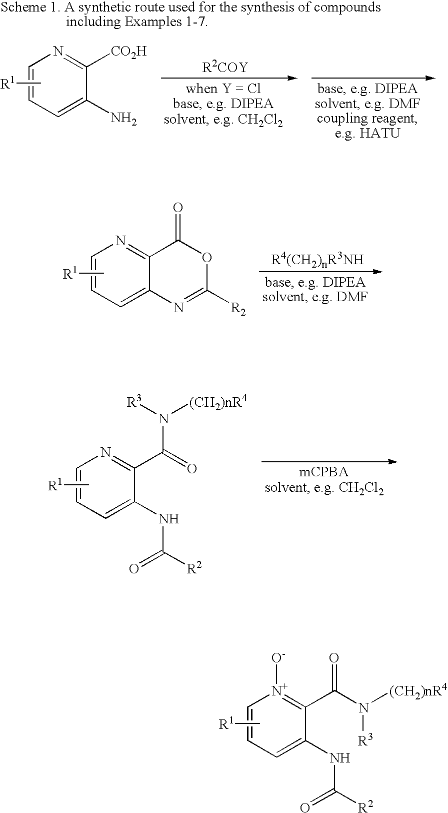 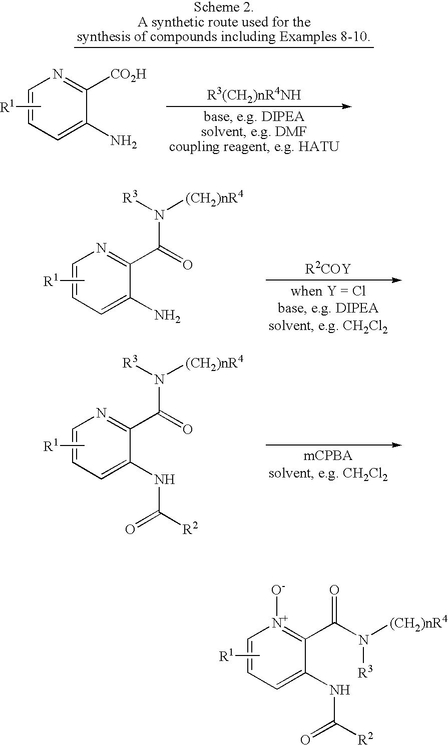
Biological Evaluation
hCB₁ and hCB₂ Receptor Binding

Human CB₁ receptor from Receptor Biology (hCB₁) or human CB₂ receptor from BioSignal (hCB2) membranes are thawed at 37° C., passed 3 times through a 25-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed in 96-well plates. The IC₅₀ of the compounds of the invention at hCB₁ and hCB₂ are evaluated from 10-point dose-response curves done with ³H-CP55,940 at 20000 to 25000 dpm per well (0.17-0.21 nM) in a final volume of 300 μl. The total and non-specific binding are determined in the absence and presence of 0.2 μM of HU210 respectively. The plates are vortexed and incubated for 60 minutes at room temperature, filtered through Unifilters GF/B (presoaked in 0.1% polyethyleneimine) with the Tomtec or Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

hCB₁ and hCB₂ GTPγS Binding

Human CB₁ receptor from Receptor Biology (hCB₁) or human CB₂ receptor membranes (BioSignal) are thawed at 37° C., passed 3 times through a 25-gauge blunt-end needle and diluted in the GTPγS binding buffer (50 mM Hepes, 20 mM NaOH, 100 mM NaCl, 1 mM EDTA, 5 mM MgCl₂, pH 7.4, 0.1% BSA). The EC₅₀ and Eₘₐₓ of the compounds of the invention are evaluated from 10-point dose-response curves done in 300 μl with the appropriate amount of membrane protein and 100000-130000 dpm of GTPg³⁵S per well (0.11-0.14 nM). The basal and maximal stimulated binding is determined in absence and presence of 1 μM (hCB₂) or 10 μM (hCB₁) Win 55,212-2 respectively. The membranes are pre-incubated for 5 minutes with 56.25 μM (hCB2) or 112.5 μM (hCB₁) GDP prior to distribution in plates (15 μM (hCB₂) or 30 μM (hCB₁) GDP final). The plates are vortexed and incubated for 60 minutes at room temperature, filtered on Unifilters GF/B (presoaked in water) with the Tomtec or Packard harvester using 3 ml of wash buffer (50 mM Tris, 5 mM MgCl₂, 50 mM NaCl, pH 7.0). The filters are dried for 1 hour at 55° C. The radioactivity (cpm) is counted in a TopCount (Packard) is after adding 65 μl/well of MS-20 scintillation liquid. Antagonist reversal studies are done in the same way except that (a) an agonist dose-response curve is done in the presence of a constant concentration of antagonist, or (b) an antagonist dose-response curve is done in the presence of a constant concentration of agonist.

Based on the above assays, the dissociation constant (Ki) for a particular compound of the invention towards a particular receptor is determined using the following equation:

Ki=IC₅₀/(1+[rad]/Kd),

Wherein IC₅₀ is the concentration of the compound of the invention at which 50% displacement has been observed;

[rad] is a standard or reference radioactive ligand concentration at that moment; and

Kd is the dissociation constant of the radioactive ligand towards the particular receptor.

Using the above-mentioned assays, the Ki towards human CB₁ receptors for most compounds of the invention is measured to be in the range of 14800 nM. The Ki towards human CB₂ receptors for most compounds of the invention is measured to be in the range of about 37-1843 nM. The EC₅₀ towards human CB₁ receptors for most compounds of the invention is measured to be in the range of about 149-2800 nM. The Eₘₐₓ towards human CB₁ receptors for most compounds of the invention is measured to be in the range of about 105-128%.

The following table shows certain biological activities for some of the exemplified compounds.

| | | Ki | EC50 | Emax |
|---|---|---|---|---|
| | | (hCB1) | (hCB1) | (hCB1) |
| COMPOUD | Structures | (nM) | (nM) | (%) |
| Example 2 | 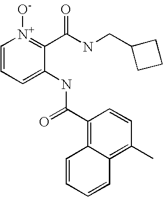 | 18 | 167 | 106 |
| Example 8 | 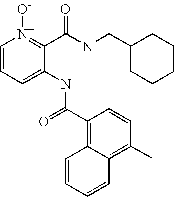 | 42 | 191 | 122 |
| Example 7 | 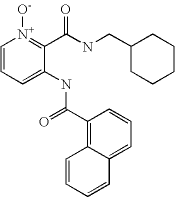 | 47 | 506 | 128 |

### EXAMPLES

The invention will further be described in more detail by the following Examples which describe methods whereby compounds of the present invention may be prepared, purified, analyzed and biologically tested, and which are not to be construed as limiting the invention.

### Example 1

N-(Cyclobutylmethyl)-3-[(1-naphthalenylcarbonyl)amino]-1-oxide-2-pyridinecarboxamide 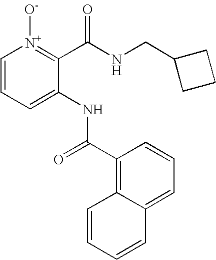
Step A. N-(Cyclobutylmethyl)-3-[(1-naphthalenylcarbonyl)amino]-1-oxide-2-pyridinecarboxamide

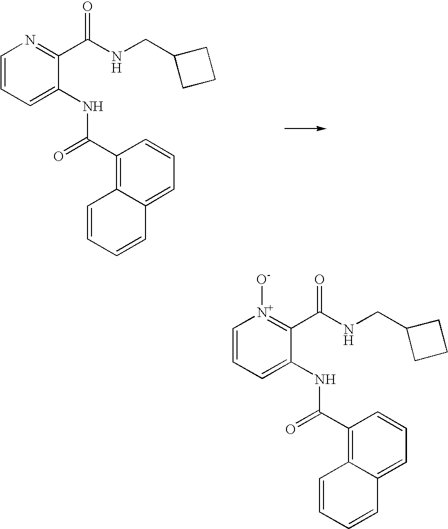

N-(Cyclobutylmethyl)-3-[(1-naphthalenylcarbonyl)amino]-2-pyridinecarboxamide (156 mg, 0.422 mmol, see Steps B & C for its preparation) in CH₂Cl₂ (10 mL) was treated with 3-chloroperoxybenzoic acid (500 mg, 1.65 mmol) for 48 h at room temperature. The mixture was diluted with CH₂Cl₂ (100 mL), washed with 1 N NaOH (2×10 mL) and dried over Na₂SO₄. After filtration and concentration, the crude products were purified by MPLC using Hex/EtOAc (1:1) on SiO₂ to provide the title compound as a white solid (114 mg, 83%). ¹H NMR (400 MHz, CDCl₃) δ 1.75 (m, 2 H), 1.89 (m, 2 H), 2.10 (m,2 H), 2.61 (m, 1 H), 3.45 (dd, J=7.23, 5.47 Hz, 2 H), 7.41 (dd, J=8.79, 6.44 Hz, 1 H), 7.57 (m, 3 H), 7.90 (m, 2 H), 8.00 (d, J=8.40 Hz, 1 H), 8.09 (dd, J=6.44, 1.17 Hz, 1 H), 8.52 (dd, J=8.20, 0.98 Hz, 1 H), 9.23 (dd, J=8.89, 1.27 Hz, 1 H), 12.15 (s, 1 H), 13.74 (s, 1 H). MS (ESI) (M+H)⁺ 376.3. Anal. Calcd for C₂₂H₂₁N₃O₃+0.1 H₂O: C, 70.05; H, 5.66; N, 11.14. Found: C, 70.00; H, 5.75; N, 11.17.
Step B. 2-(1 -Naphthalenyl)-H-pyrido[3,2-d] [1,3]oxazin-4-one

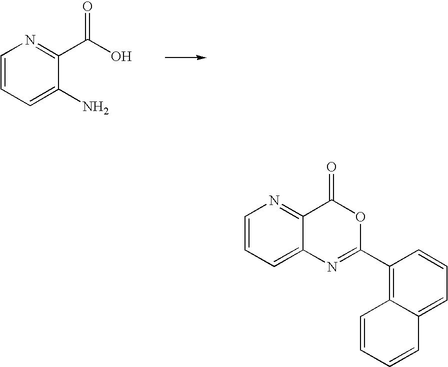

1-Naphthalenecarbonyl chloride (400 mg, 2.1 mmol) in CH₂Cl₂ (2 mL) was added into a solution of 3-amino-2-pyridinecarboxylic acid (277 mg, 2.0 mmol) and diisopropylethylamine (284 mg, 2.2 mmol) in DMF (10 mL) at 0° C. The reaction mixture was allowed to stir overnight at room temperature, and was then treated with diisopropylethylamine (284 mg, 2.2 mmol) and HATU (837 g, 2.2 mmol). After stirring for 1 h at room temperature, the reaction mixture was heated at 50° C. to provide the title compound which was used in Step A. MS (ESI) (M+H)⁺ 274.79.
Step C. N-(Cyclobutylmethyl)-3-[(1-naphthalenylcarbonyl)amino]-2-pyridinecarboxamide

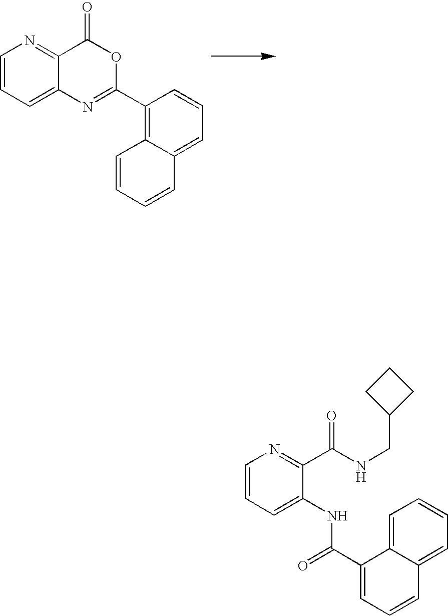

A solution of 2-(1-naphthalenyl)-H-pyrido[3,2-d][1,3]oxazin-4-one (100 mg, 0.365 mmol, see Step B for its preparation) in DMF (2 mL) was treated with cyclobutane methylamine (0.1 mL, 5.3 M in MeOH, 0.53 mmol) at 0° C. The mixture was stirred for 18 h at room temperature. After evaporation of the solvent, the residue was purified by MPLC using Hex/EtOAc (9:1) on siO₂ to provide the title compound (156 mg, 83%). ¹H NMR (400 MHz, CDCl₃) 6 1.69-1.78 (m, 2 H), 1.81-1.91 (m, 2 H), 1.99-2.07 (m, 2 H), 2.51-2.62 (m, 1 H), 3.34 (d, J=7.03 Hz, 2 H), 7.52-7.59 (m, 4 H), 7.87-7.89 (m, 1 H), 7.92-7.96 (m, 1 H), 8.03-8.05 (m, 1 H), 8.30-8.35 (m, 1 H), 8.42-8.45 (m, 1 H), 9.27 (dd, J=8.59, 1.17 Hz, 1 H). MS (ESI) (M+H)⁺ 360.0. Anal. (C, H, N) calcd for C₂₂H₂₁N₃O₂+0.3 CH₃OH: C 72.58, H 6.06, N 11.39; found C 72.58, H 5.86, N 11.30.

### Example 2

N-(Cyclobutylmethyl)-3-[[(4-methyl-1-naphthalenyl)carbonyl]amino]-1-oxide-2-pyridinecarboxamide 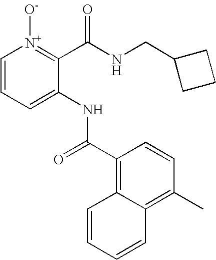
Step A. N-(Cyclobutylmethyl)-3-[[(4-methyl-1-naphthalenyl)carbonyl]amino]-1-oxide-2-pyridinecarboxamide

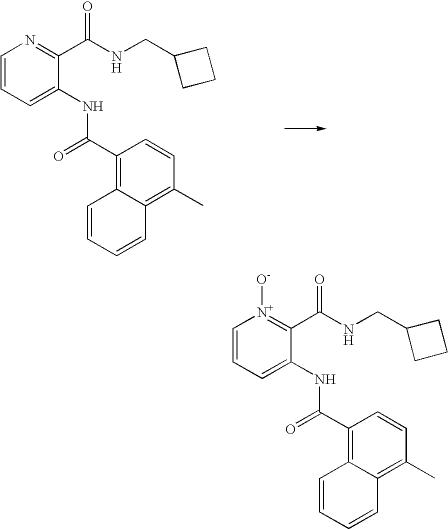

Following the procedure for Step A in Example 1, N-(cyclobutylmethyl)-3-[[(4-methyl-1-naphthalenyl)carbonyl]amino]-2-pyridinecarboxamide (105 mg, 0.279 mmol, see Steps B & C for its preparation) in CH₂Cl₂ (10 mL) was treated with 3-chloroperoxybenzoic acid (422 mg, 1.4 mmol). The crude products were purified by reverse phase HPLC using 60-85% MeCN/H₂O to provide the title compound as a white solid (45 mg, 41 %). H NMR (400 MHz, CD₃0D) δ 1.77 (m, 2 H), 1.88 (m, 2 H), 2.05 (m, 2 H), 2.59 (m, 1 H), 2.75 (s, 3 H), 3.41 (d, J=7.03 Hz, 2 H), 7.44 (d, J=7.62 Hz, 1 H), 7.59 (m, 3 H), 7.77 (d, J=7.23 Hz, 1 H), 8.13 (m, 1 H), 8.19 (dd, J=6.44, 0.78 Hz, 1 H), 8.45 (m, 1 H), 9.04 (dd, J=8.79, 0.98 Hz, 1 H). MS (ESI) (M+H)⁺ 390.0.
Step B. 2-(4-Methyl-1-naphthalenyl)-4H-pyrido[3,2-d][1,3]oxazin-4-one

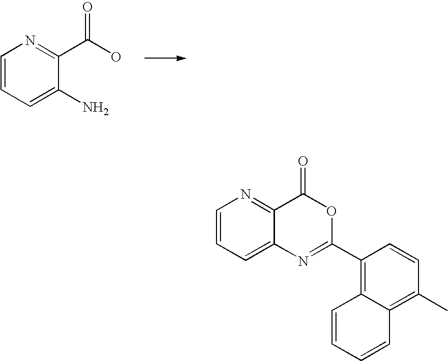

Following the procedure for Step B in Example 1, a suspension of 3-amino-2-pyridinecarboxylic acid (414 mg, 3.0 mmol) in CH₂Cl₂ (10 mL) and diisopropylethylamine (1.25 mL, 7.2 mmol) was treated with 4-methyl-1-naphthalenecarbonyl chloride, prepared from 4-methyl-1-naphthalenecarbonylic acid (590 mg, 3.17 mmol) with thionyl chloride (4.11 g, 35 mmol), and then with HATU (1.25 g, 3.3 mmol) in DMF (10 mL). The title compound was formed and directly used in Step C.
Step C. N-(Cyclobutylmethyl)-3-[[(4-methyl-1-naphthalenyl)carbonyl]amino]-2-pyridinecarboxamide

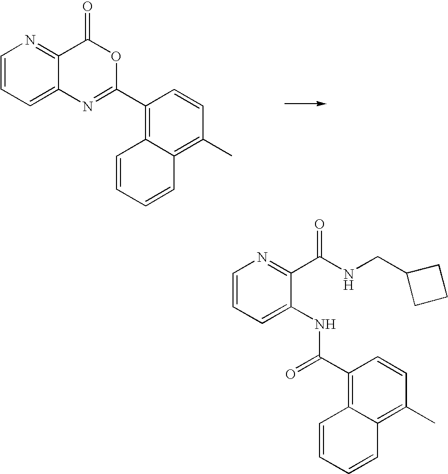

Following the procedure for Step C in Example 1, using 2-(4-methyl-1-naphthalenyl)-4H-pyrido[3,2-d][1,3]oxazin-4-one (130 mg, 0.45 mmol, see Step B for its preparation) and cyclobutylmethylamine (0.5 mL, 5.3 Min MeOH, 2.5 mmol) provided the title compound (105 mg, 72%). ¹H NMR (400 MHz, CD₃0D) 6 1.77 (m, 2 H), 1.87 (m, 2 H), 2.05 (m, 2 H), 2.60 (m, 1 H), 2.76 (s, 3 H), 3.37 (d, J=7.03 Hz, 2 H), 7.46 (d, J=7.23 Hz, 1 H), 7.59 (m, 3 H), 7.80 (d, J=7.23 Hz, 1 H), 8.14 (m, 1 H), 8.36 (dd, J=4.49, 1.37 Hz, 1 H), 8.46 (m, 1 H), 9.29 (dd, J=8.59, 1.37 Hz, 1 H). MS (ESI) (M+H)⁺ 374.0.

### Example 3

N-(Cyclobutylmethyl)-3-[[(4-methoxy- 1 -naphthalenyl)carbonyl]amino]-1-oxide-2-pyridinecarboxamide 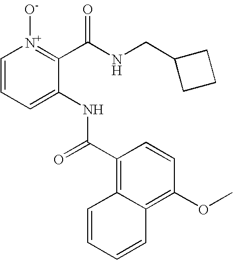
Step A. N-(Cyclobutylmethyl)-3-[[(4-methoxy-1-naphthalenyl)carbonyl]amino]-1-oxide-2-pyridinecarboxamide

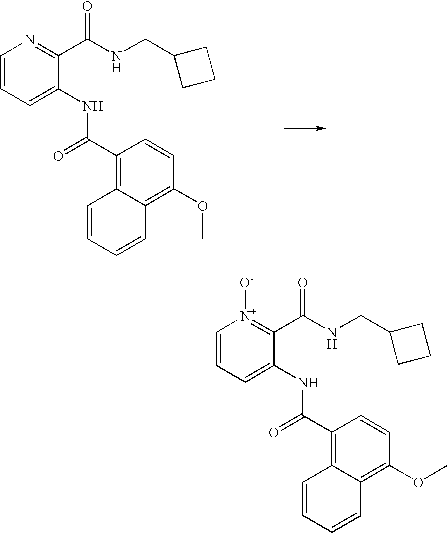

Following the procedure for Step A in Example 1, N-(cyclobutylmethyl)-3-[[(4-methoxy-1-naphthalenyl)carbonyl]amino]-2-pyridinecarboxamide (87 mg, 0.224 mmol, see Steps B & C for its preparation) in CH₂Cl₂ (10 mL) was treated with 3-chloroperoxybenzoic acid (400 mg, 1.32 mmol). The crude product was purified by reverse phase HPLC using 55-80% MeCN/H₂O to provide the title compound as a white solid (11 Img, 12 %). ¹H NMR (400 MHz, CD₃OD) δ 1.79 (m, 2 H), 1.88 (m, 2 H), 2.06 (m, 2 H), 2.61 (m, 1 H), 3.43 (d, J=7.03 Hz, 2 H), 4.08 (s, 3 H), 7.01 (d, J=8.01 Hz, 1 H), 7.56 (m, 3 H), 7.91 (d, J=8.20 Hz, 1 H), 8.18 (dd, J=6.35, 1.07 Hz, 1 H), 8.31 (m, 1 H,) 8.51 (d, J=8.20 Hz, 1 H), 9.04 (dd, J=8.79, 1.17 Hz, 1 H). MS (ESI) (M+H)+406.0.
Step B. 2-(4-Methoxy-1-naphthalenyl)-4H-pyrido[3,2-d][1,3]oxazin-4-one

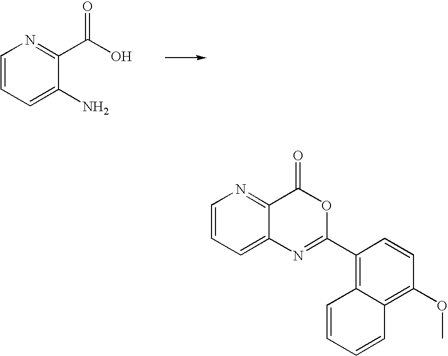

Following the procedure for Step B in Example 1, using 3-amino-2-pyridinecarboxylic acid (690 mg, 5.0 mmol), diisopropylethylamine (780 mg, 6.0 mmol), 4-methoxy-1-naphthalenecarbonyl chloride, prepared from 4-methoxy-1-naphthoic acid (1.0 g, 5.0 mmol) and oxalyl chloride (5 mL, 2.0 M in CH₂Cl₂, 10 mmol), and then HATU (2.28 g, 6.0 mmol) provided the title compound which was directly used in Step C.
Step C. N-(Cyclobutylmethyl)-3-[[(4-methoxy-1-naphthalenyl)carbonyl]amino]-2-pyridinecarboxamide

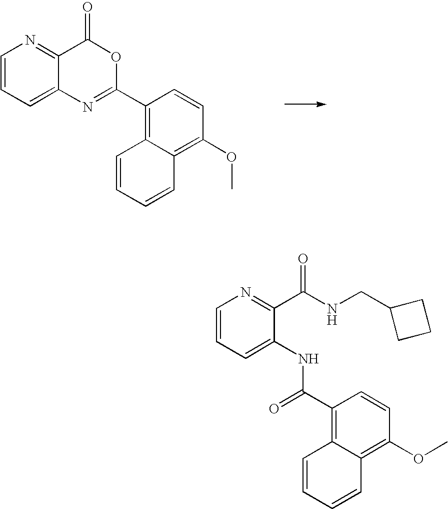

Following the procedure for Step C in Example 1, using 2-(4-methoxy-1-naphthalenyl)-4H-pyrido[3,2-d][1,3]oxazin-4-one (120 mg, 0.40 mmol, see Step B for its preparation) and cyclobutylmethylamine (0.5 mL, 5.3 Min MeOH, 2.5 mmol) provided the title compound (87 mg, 56 %). ¹H NMR (400 MHz, CD₃0D) 6 1.77 (m, 2 H), 1.88 (m, 2 H), 2.06 (m, 2 H), 2.61 (m, 1 H), 3.38 (d, J=7.23 Hz, 2 H), 4.08 (s, 3 H), 7.02 (d, J=8.20 Hz, 1 H), 7.56 (m, 3 H), 7.93 (d, J=8.01 Hz, 1 H), 8.32 (m, 2 H), 8.52 (m, 1 H), 9.27 (dd, J=8.59, 1.37 Hz, 1 H). MS (ESI) (M+H)+390.0.

### Example 4

3-[(1 -Naphthalenylcarbonyl)amino]-N-[(tetrahydro-2H-pyran-4-yl)methyl]-1 -oxide-2-pyridinecarboxamide 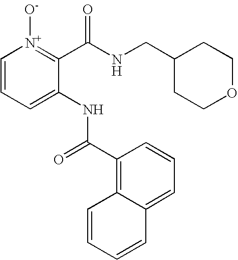
Step A. 3 -[(1 -Naphthalenylcarbonyl)amino]-N-[(tetrahydro-2H-pyran4-yl)methyl]-1-oxide-2-pyridinecarboxamide

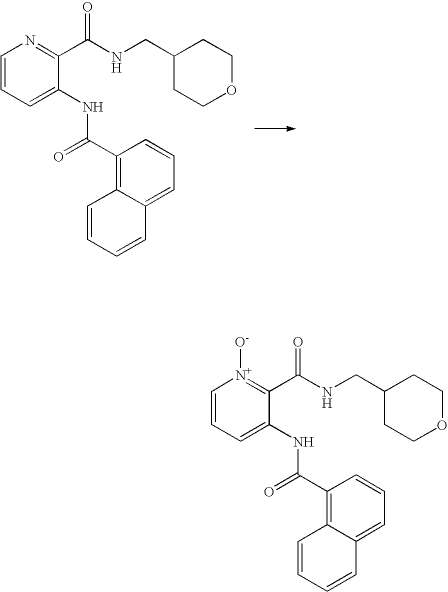

Following the procedure for Step A in Example 1, 3-[(1-naphthalenylcarbonyl)amino]-N-[(tetrahydro-2H-pyran-4-yl)methyl]-2-pyridinecarboxamide (139 mg, 0.356 mmol, see Step B for its preparation) in CH₂Cl₂ (10 mL) was treated with 3-chioroperoxybenzoic acid (323 mg, 1.07 rnrol). The crude product was purified by MPLC using Hex/EtOAc (1: 1) on SiO₂ to provide the title compound as a white solid (100 nig, 63 %). ¹H NMR (400 MHz, DMSO-D₆) 6 1.14 (m, 2 H), 1.52 (dd, J=12.79, 1.86 Hz, 2 H), 1.71 (m, 1 H), 3.16 (m, 4 H), 3.74 (dd, J=11.13, 3.71 Hz, 2 H), 7.60 (m, 4 H), 7.84 (d, J=7.23 Hz, 1 H), 8.02 (m, 1 H), 8.12 (d, J=8.20 Hz, 1 H), 8.26 (m, 1 H), 8.32 (mi, 1 H), 8.72 (m, 1 H), 11.54 (s, 1 H), 12.81 (s, 1 H), 12.87 (s, 1 H). MS (ESI) (M+H)⁺ 406.0. Anal. (C, H. N) calcd for C₂₃H₂₃N₃O₄+0.1 HC1: C 67.53, H 5.69, N 10.27; found C 67.43, H 5.63, N 10.04.
Step B. 3 -[(1 -Naphthalenylcarbonyl)amino]-N-[(tetrahydro-2H-pyran-4-yl)methyl]-2-pyridinecarboxamide

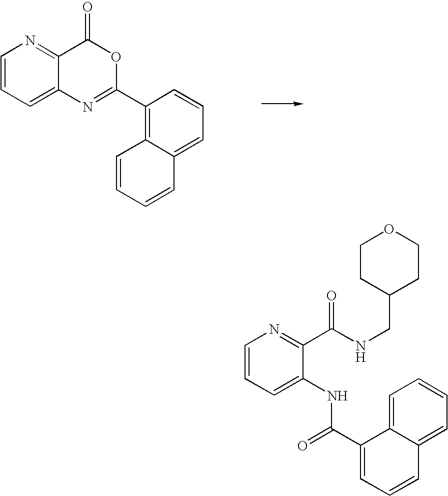

Following the procedure for Step C in Example 1, using 2-(1-naphthalenyl)-4H-pyrido[3,2-d][1,3]oxazin-4-one (122 mg, 0.446 mmol) and tetrahydro-2H-pyran-4-methanamine (62 mg, 0.535 mmol) provided the title compound (139 mg, 90 %). HH NMR (400 MHz, CDCl₃) δ 0.98 (m, 2H), 1.23 (m, 3H), 1.56 (m, 1H), 1.76 (m, SH), 3.25 (t, J =6.4 Hz, 2H), 7.54 (m, 4H), 7.90 (m, 2H), 7.98 (d, J =8.0 Hz, IH), 8.28 (dd, J =8.4, 1.6 Hz, 1H), 8.53 (m, 2H), 9.41 (dd, J=8.4, 0.8 Hz, 1H), 12.87 (s, 1H)., MS (ESI) (M+H)⁺ 390.2; Anal. Calcd for C₂₃H₂₃N₃O₃: C, 70.93; H, 5.95; N, 10.79. Found: C, 70.82; H, 5.92; N, 10.64.

### Example 5

3-[[(4-Methyl-1-naphthalenyl)carbonyl]amino]-N-[(tetrahydro-2H-pyran-4-yl)methyl]-1-oxide-2-pyridinecarboxamide 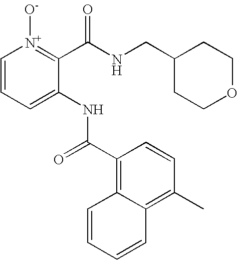
Step A. 3-[[(4-Methyl-1-naphthalenyl)carbonyl]amino]-N-[(tetrahydro-2H-pyran-4-yl)methyl]-1-oxide-2-pyridinecarboxamide

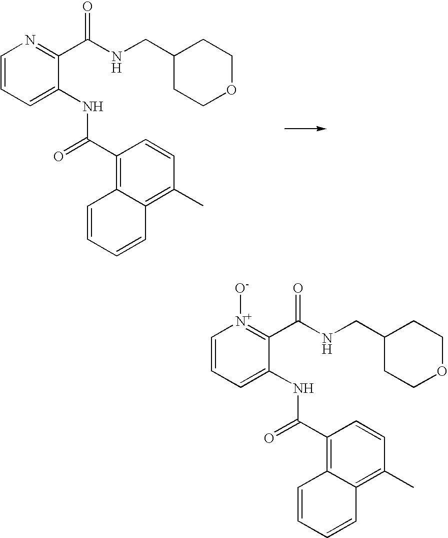

Following the procedure for Step A in Example 1, 3-[[(4-methyl-1-naphthalenyl)carbonyl]amino]-N-[(tetrahydro-2H-pyran-4-yl)methyl]-2-pyridinecarboxamide (38.8 mg, 0.096 mmol, see Step B for its preparation) in CH₂Cl₂ (10 mL) was treated with 3-chioroperoxybenzoic acid (220 mg, 0.73 mmol). The crude product was purified by MPLC using EtOAc on SiO₂ to provide the title compound as a white solid (30 mg, 76 %). ¹H NMR (400 MHz, CD₃OD) δ 1.30 (mi, 2 H), 1.65 (d, J=13.08 Hz, 2 H), 1.83 (m, 1 H), 2.76 (s, 3 H), 3.34 (m, 4 H), 3.88 (dd, J=l 1.13, 3.51 Hz, 2 H), 7.44 (d, J=7.23 Hz, 1 H), 7.59 (mi, 3 H), 7.77 (d, J=7.23 Hz, 1 H), 8.14 (m, 1 H), 8.21 (dd, J=6.44, 0.98 Hz, 1 H), 8.44 (m, 1 H), 8.99 (dd, J=8.79, 0.98 Hz, 1 H). MS (ESI) (M+H)⁺ 420.0. Anal. (C, H, N) calcd for C₂₄H₂₅N₃O₄+0.3 H₂O: C 67.85, H 6.07, N 9.89; found C 67.87, H 5.88, N 9.80.
Step B. 3-[[(4-Methyl-1-naphthalenyl)carbonyl]amino]-N-[(tetrahydro-2H-pyran-4-yl)methyl]-2-pyridinecarboxamide

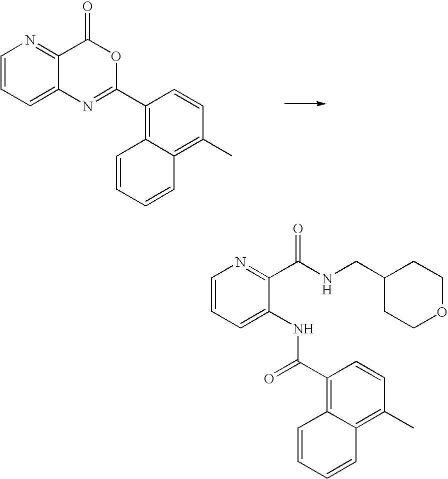

Following the procedure for Step C in Example 1, using 2-(4-methyl-1-naphthalenyl)-4H-pyrido[3,2-d][1,3]oxazin-4-one (108 mg, 0.375 mmol) and tetrahydro-2H-pyran-4-methanamine (122 mg, 1.06 mmol) provided the title compound (75 mg, 49%). ¹H NMR (400 MHz, CD₃OD) δ 1.26 (dd, J=11.91, 4.49 Hz, 1 H), 1.33 (dd, J=11.9,4.5 Hz, 1 H), 1.63 (m, 2 H), 1.85 (m, 1 H), 2.76 (s, 3 H), 3.24 (d, J=7.03 Hz, 2 H), 3.36 (m, 2 H), 3.90 (dd, J=11.03, 3.22Hz, 2 H), 7.45 (m, 1 H), 7.60 (m, 3 H), 7.79 (d, J=7.23 Hz, I H), 8.13 (m, 1 H), 8.36 (dd, J=4.49, 1.37 Hz, 1 H), 8.46 (m, 1 H), 9.28 (dd, J=8.59, 1.37 Hz, 1 H). MS (ESI) (M+H)⁺ 404.0. Anal. (C, H, N) calcd for C₂₄H₂₅N₃O₃+0.1 H₂O: C 71.13, H 6.27, N 10.37; found C 71.03, H 6.04, N 10.26.

### Example 6

3-[[(4-Methoxy-1-naphthalenyl)carbonyl]amino]-N-[(tetrahydro-2H-pyran-4-yl)methyl]-1-oxide-2-pyridinecarboxamide 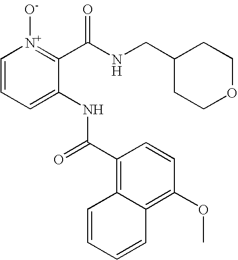
Step A. 3-[[(4-Methoxy-1-naphthalenyl)carbonyl]amino]-N-[(tetrahydro-2H-pyran-4-yl)methyl] 1 -oxide-2-pyridinecarboxamide

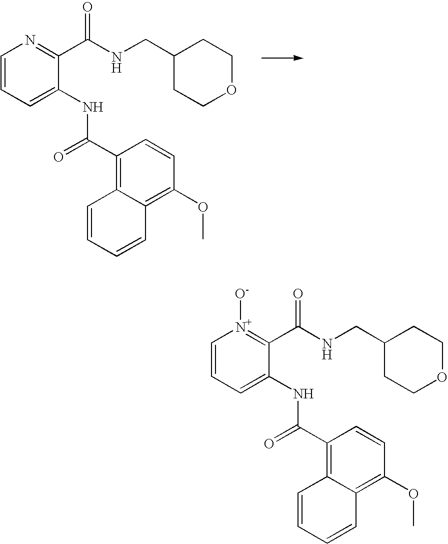

Following the procedure for Step A in Example 1, 3-[[(4-methoxy-1-naphthalenyl)carbonyl]amino]-N-[(tetrahydro-2H-pyran-4-yl)methyl]-2-pyridinecarboxamide (78.8 mg, 0.188 mmol, see Step B for its preparation) in CH₂Cl₂ (10 mL) was treated with 3-chloroperoxybenzoic acid (252 mg, 1.13 mmol). The crude product was purified by reverse phase HPLC using 30-80% MeCN/H₂O to provide the title compound as a white solid (21 mg, 20%). ¹H NMR (400 MHz, CD₃OD) δ 1.32 (m, 2 H), 1.66 (m, J=12.69 Hz, 2 H), 1.85 (m, 1 H), 3.33 (m, 4 H), 3.88 (dd, J=11.13, 3.71 Hz, 2 H), 4.08 (s, 3 H), 7.01 (d, J=8.20 Hz, 1 H), 7.56 (m, 3 H), 7.91 (d, J=8.20 Hz, 1 H), 8.19 (d, J=6.44 Hz, 1 H), 8.31 (d, J=8.20 Hz, 1 H), 8.50 (d, J=8.40 Hz, 1 H), 8.99 (d, J=8.79 Hz, 1 H). MS (ESI) (M+H)+436.0.
Step B. 3-[(4-Methoxy-1-naphthoyl)amino]-N-(tetrahydro-2H-pyran-4-ylmethyl)pyridine-2-carboxamide

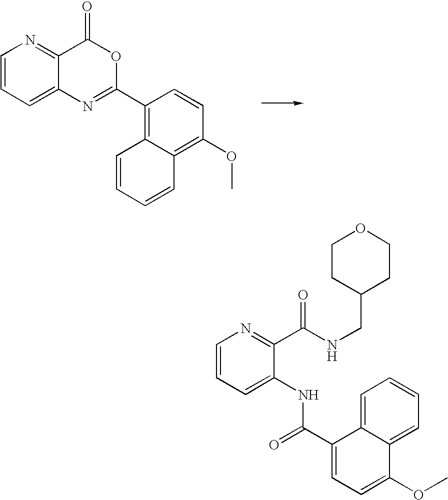

Following the procedure for Step C in Example 1, using 2-(4-methoxy-1-naphthyl)-4H-pyrido[3,2-d][1,3]oxazin-4-one (120 mg, 0.4 mmol), and tetrahydro-2H-pyran-4-methanamine (210 mg, 1.8 mmol) provided the title compound (81 mg, 48 %). ¹H NMR (400 MHz, CD₃OD) δ 1.31 (m, 2 H), 1.64 (dd, J=13.08, 1.17 Hz, 2 H), 1.87 (m, J=7.62, 3.51 Hz, 1 H), 3.26 (m, J=6.83 Hz, 2 H), 3.36 (m, 2 H), 3.91 (dd, J=11.72, 3.51 Hz, 2 H), 4.08 (s, 3 H), 7.01 (d, J=8.20 Hz, 1 H), 7.56 (m, 3 H), 7.93 (d, J=8.01 Hz, 1 H), 8.33 (m, 2 H), 8.51 (d, J=8.59 Hz, 1 H), 9.26 (m, 1 H). MS (ESI) (M+H)⁺ 420.0

### Example 7

N-(Cyclohexylmethyl)-3-[(1-naphthalenylcarbonyl)amino]-1-oxide-2-pyridinecarboxamide 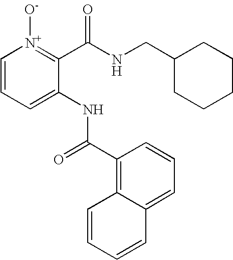
Step A. N-(Cyclohexylmethyl)-3-[(1-naphthalenylcarbonyl)amino]-1-oxide-2-pyridinecarboxamide

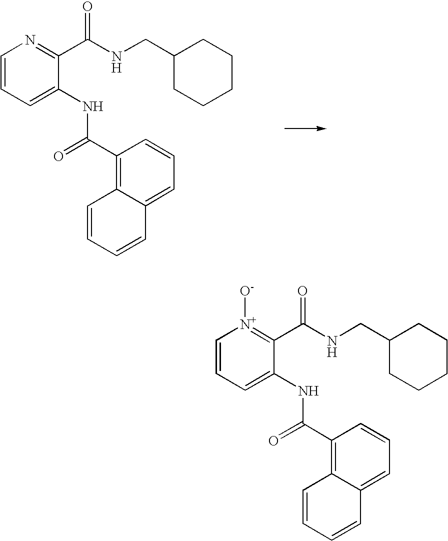

Following the procedure for Step A in Example 1, N-(cyclohexylmethyl)-3-[(1-naphthalenylcarbonyl)amino]-2-pyridinecarboxamide (172 mg, 0.45 mmol, see Step B for its preparation) in CH₂Cl₂ (10 mL) was treated with 3-chloroperoxybenzoic acid (295 mg, 1.3 mmol). The crude product was purified by reverse phase HPLC using 30-80% MeCN/H₂O to provide the title compound as a white solid (161 mg, 89 %). ¹H NMR (400 MHz, CD₃OD) δ 1.00 (m, 2 H), 1.19 (m, 3 H), 1.56 (m, 1 H), 1.70 (m, 5 H), 3.23 (d, J=6.64 Hz, 2 H), 7.58 (m, 4 H), 7.88 (dd, J=7.13, 1.07 Hz, 1 H), 7.96 (m, 1 H), 8.07 (d, J=8.20 Hz, 1 H), 8.21 (d, J=6.44 Hz, 1 H), 8.42 (m, 1 H), 9.03 (d, J=8.79 Hz, 1 H). MS (ESI) (M+H)⁺ 404.0.
Step B. N-(Cyclohexylmethyl)-3-[(1-naphthalenylcarbonyl)amino]-2-pyridinecarboxamide

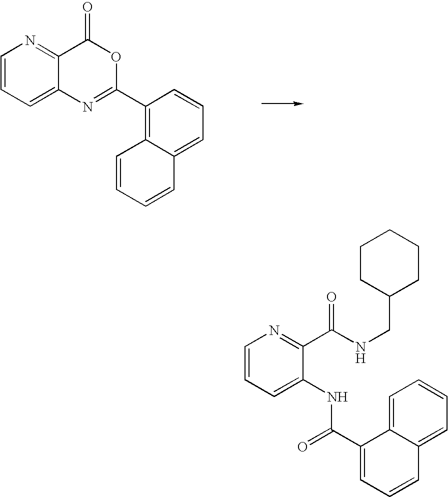

Following the procedure for Step C in Example 1, using 2-(1-naphthalenyl)-4H-pyrido[3,2-d][1,3]oxazin-4-one (129 mg, 0.47 mmol), and cyclohexanemethylamine (261 mg, 2.3 mmol) provided the title compound (172 mg, 95 %). ¹H NMR (400 MHz, CD₃OD) δ 0.90-1.00 (m, 2 H), 1.13-1.28 (m, 3 H), 1.52-1.75 (m, 6 H), 3.16 (d, J=6.83 Hz, 2 H), 7.55-7.61 (m, 4 H), 7.88-7.90 (m, 1 H), 7.94-7.96 (m, 1 H), 8.05-8.07 (m, 1 H), 8.36 (dd, J=4.49, 1.56Hz, 1 H), 8.41-8.43 (m, 1 H), 9.29 (dd, J=8.59, 1.37 Hz, 1 H). MS (ESI) (M+H)⁺ 388.0.

### Example 8

N-(Cyclohexylmethyl)-3-[(4-methyl-1-naphthalenylcarbonyl)amino]-1-oxide-2-pyridinecarboxamide 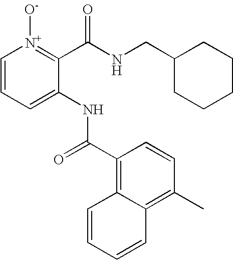
Step A. N-(Cyclohexylmethyl)-3-[(4-methyl-1-naphthalenylcarbonyl)amino]-1-oxide-2-pyridinecarboxamide

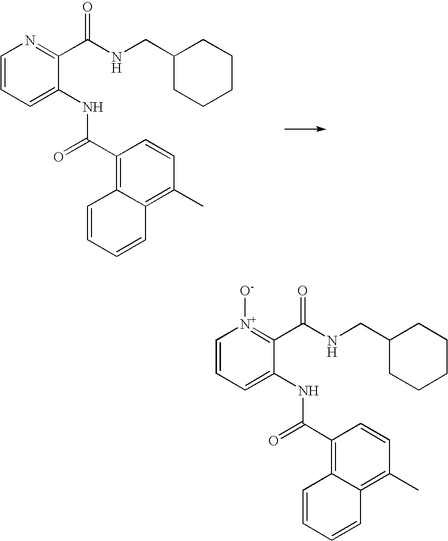

Following the procedure for Step A in Example 1, N-(cyclohexylmethyl)-3-[(4-methyl-1-naphthalenylcarbonyl)amino]-2-pyridinecarboxamide (66 mg, 0.165 mmol, see Step B & C for its preparation) in CH₂Cl₂ (10 mL) was treated with 3-chloroperoxybenzoic acid (111.0 mg, 0.495 mmol). The crude product was purified by reverse phase HPLC using 30-80% MeCN/H₂O to provide the title compound as a white solid (56 mg, 81 %). ¹H NMR (400 MHz, CD₃OD) δ 0.91-1.06 (m, 2 H), 1.12-1.32 (m, 3 H), 1.48-1.62 (m, 1 H), 1.60-1.82 (m, 5 H), 2.75 (s, 3 H), 3.23 (d, J=6.83 Hz, 2 H), 7.44 (d, J=7.22 Hz, 1 H), 7.53-7.64 (m, 3 H), 7.77 (d, J=7.23 Hz, 1 H), 8.08-8.15 (m, 1 H), 8.19 (dd, J=6.44, 1.17 Hz, 1 H), 8.41-8.47 (m, 1 H), 9.02 (dd, J=8.79, 1.17 Hz, 1 H). MS (ESI) (M+H)⁺ 418.0
Step B. 3-Amino-N-(cyclohexylmethyl)pyridine-2-carboxamide

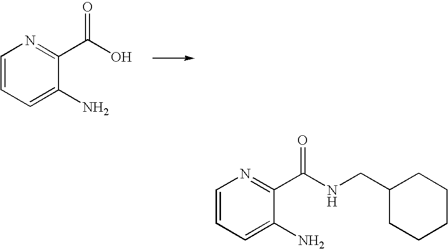

3-Aminopyridine-2-carboxylic acid (138 mg, 1.0 mmol) was added to a solution of cyclohexane methylamine (226 mg, 2.0 mmol) and DIPEA (259 mg, 0.35 mmol) in DMF (5 mL). After stirring for 30 min, HATU (456 mg, 1.2 mmol) was added at 0° C. The resulting mixture was stirred overnight at room temperature, quenched with water (5 ml), concentrated to a small volume, diluted with EtOAc (100 mL), washed with water (2×5 mL) and brine (5 nmL), then dried with sodium sulphate. After filtration and concentration, the crude product was purified by MPLC using Hex/EtOAc (1:1) on SiO₂ to provide the title compound (124 mg, 53%). ¹H NMR (400 MHz, CDCl₃) δ 0.93-1.07 (m, 2 H), 1.13-1.32 (m, 3 H), 1.51-1.70 (m, 2 H), 1.70-1.86 (m, 4 H), 3.26 (t, J=6.64 Hz, 2 H), 6.00 (s, 2 H), 7.00 (dd, J=8.40, 1.37 Hz, 1 H), 7.15 (dd, J=8.40, 4.30 Hz, 1 H), 7.85 (dd, J=4.30, 1.37 Hz, 1 H), 8.22 (s, 1 H). (MS (ESI) (M+H)⁺ 233.89.
Step C. N-(Cyclohexylmethyl)-3-[(4-methyl-1-naphthalenylcarbonyl)amino]-2-pyridinecarboxamide

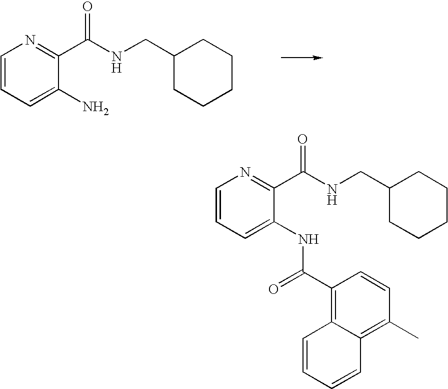

4-Methyl-1-naphthalenecarbonyl chloride (80 mg, 0.39 mmol) was added to a solution of 3-amino-N-(cyclohexylmethyl)pyridine-2-carboxamide (61 mg, 0.26 mmol) and DMAP (64 mg, 0.52 mmol) in CH₂Cl₂ (10 mL) at 0° C. The mixture was stirred overnight at room temperature, quenched with saturated NaHCO₃ (5 mL), and extracted with EtOAc (3×50 mL). The combined oranic phase was washed with brine (2×10 mL) and dried with sodium sulphate. After filtration and concentration, the crude products were purifed by MPLC using hexane/EtOAc (4:1) on SiO₂ to provide the title compound as a white solid (96 mg, 92%). ¹H NMR (400 MHz, CD₃OD) δ 0.88-1.05 (m, 2 H), 1.09-1.34 (m, 3 H), 1.52-1.68 (m, 2 H), 1.68-1.81 (m, 4 H), 2.76 (s, 3 H), 3.18 (d, J=6.83 Hz, 2 H), 7.39-7.50 (m, 1 H), 7.54-7.65 (mn, 3 H), 7.80 (d, J=7.23 Hz, 1 H), 8.06-8.18 (mn, 1 H), 8.36 (dd, J=4.49, 1.56 Hz, 1 H), 8.43-8.50 (m, 1 H), 9.29 (dd, J=8.59, 1.56 Hz, 1 H). MS (ESI) (M+H)⁺ 402.0

### Example 9

N-(Cyclohexylmethyl)-3-[(4-methoxy-1-naphthalenylcarbonyl)amino]-1-oxide-2-pyridinecarboxamide 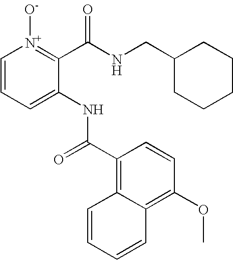
Step A. N-(Cyclohexylmethyl)-3-[(4-methoxy-1-naphthalenylcarbonyl)amino]-1-oxide-2-pyridinecarboxamide

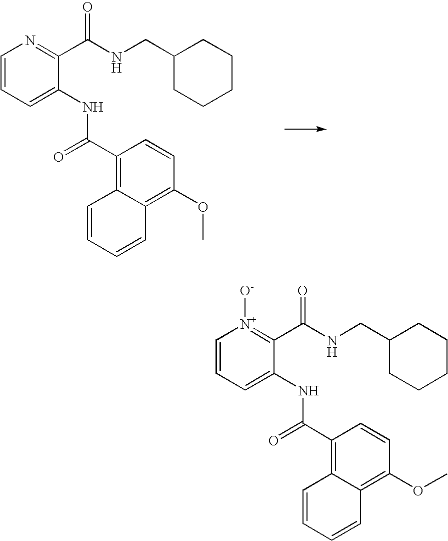

Following the procedure for Step A in Example 1, N-(cyclohexylmethyl)-3-[(4-methoxy-1-naphthalenylcarbonyl)amino]-2-pyridinecarboxamide (76 mg, 0.18 mmol, see Step B for its preparation) in CH₂Cl₂ (10 mL) was treated with 3-chloroperoxybenzoic acid (122 mg, 0.543 mmol). The crude product was purified by reverse phase HPLC using 30-80% MeCN/H₂O to provide the title compound as a white solid (15 mg, 19%). ¹H NMR (400 MHz, CD₃OD) δ 0.93-1.07 (m, 2 H), 1.13-1.30 (m, 3 H), 1.51-1.60 (m, 1 H), 1.62-1.82 (m, 5 H), 3.25 (d, J=6.64 Hz, 2 H), 4.08 (s, 3 H), 7.01 (d, J=8.20 Hz, 1 H), 7.47-7.65 (m, 3 H), 7.91 (d, J=8.20 Hz, 1 H), 8.18 (dd, J=6.44, 0.98 Hz, 1 H), 8.27-8.35 (m, 1 H), 8.48-8.56 (m, 1 H), 9.02 (dd, J=8.89, 1.07 Hz, 1 H). MS (ESI) (M+H)⁺ 434.0.
Step B. N-(Cyclohexylmethyl)-3-[(4-methoxy-1-naphthalenylcarbonyl)amino]-2-pyridinecarboxamide

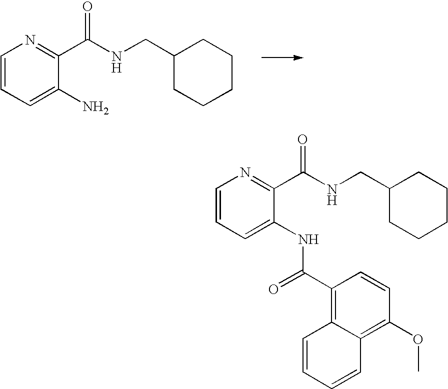

Following the procedure for Step C in Example 8, 3-amino-N-(cyclohexylmethyl)pyridine-2-carboxamide (60 mg, 0.26 mmol) was treated with 4-methoxy-1-naphthalenecarbonyl chloride (71 mg, 0.32 mmol) in CH₂Cl₂ (10 mL) in the presence of DMAP (64 mg, 0.52 mmol). The crude products were purifed by MPLC using hexane/EtOAc (4:1) on siO₂ to provide the title compound as a white solid (76 mg, 70%). ¹H NMR (400 MHz, CD₃OD) δ 0.90-1.04 (m, 2 H), 1.14-1.34 (m, 3 H), 1.60 (d, J=3.51 Hz, 2 H), 1.69-1.81 (m, 4 H), 3.20 (d, J=7.03 Hz, 2 H), 4.08 (s, 3 H), 7.02 (d, J=8.20 Hz, 1 H), 7.44-7.64 (m, 3 H, 7.93 (d, J=8.01 Hz, 1 H), 8.28-8.40 (m, 2 H), 8.51 (d, J=8.40 Hz, 1 H), 9.27 (dd, J=8.59, 1.37 Hz, 1 H). MS (ESI) (M+H)⁺ 418.0.

### Example 10

N-(Cyclohexylmethyl)-3-[(4-methoxy-1-naphthalenylcarbonyl)amino]-1-oxide-2-pyridinecarboxamide 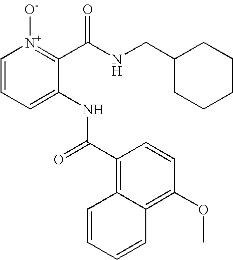
Step A. N-(Cyclohexylmethyl)-3-[(4-methoxy-1-naphthalenylcarbonyl)arnino]-1-oxide-2-pyridinecarboxamnide

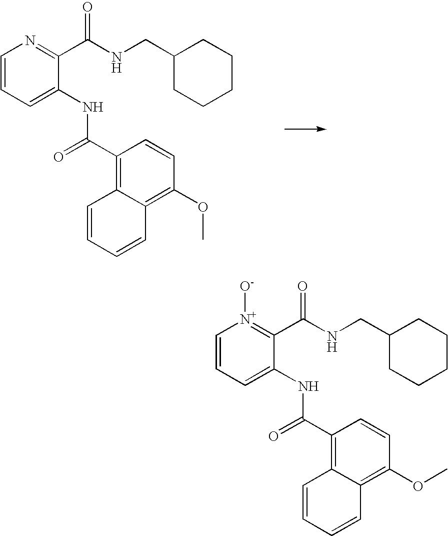

Following the procedure for Step A in Example 1, N-(cyclohexylmethyl)-3-[(4-methoxy-1-naphthalenylcarbonyl)amino]-2-pyridinecarboxamide (76 mg, 0.18 mmol, see Step B for its preparation) in CH₂Cl₂ (10 mL) was treated with 3-chloroperoxybenzoic acid (122 mg, 0.543 mmol). The crude product was purified by reverse phase HPLC using 30-80% MeCN/H₂O to provide thetitlecompoundasawhitesolid (15mg, 19%). ¹HNMR(400 MHz, CD₃OD) δ 0.93-1.07 (m, 2 H), 1.13-1.30 (m, 3 H), 1.51-1.60 (m, 1 H), 1.62-1.82 (m, 5 H), 3.25 (d, J=6.64 Hz, 2 H), 4.08 (s, 3 H), 7.01 (d, J=8.20 Hz, 1 H), 7.47-7.65 (m, 3 H), 7.91 (d, J=8.20 Hz, 1 H), 8.18 (dd, J=6.44, 0.98 Hz, 1 H), 8.27-8.35 (m, 1 H), 8.48-8.56 (m, 1 H), 9.02 (dd, J=8.89, 1.07 Hz, 1 H). MS (ESI) (M+H)⁺ 434.0. Anal. Calcd for C₂₅H₂₇N₃O₄+0.1 MeOH (436.71): C, 69.03; H. 6.32; N. 9.62. Found: C, 69.04; H 6.25; N. 9.45.
Step B. N-(Cyclohexylmethyl)-3-[(4-methoxy-1-naphthalenylcarbonyl)amino]-2-pyridinecarboxamide

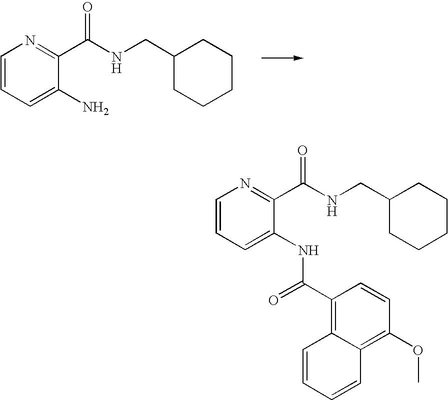

Following the procedure for Step C in Example 9, 3-amino-N-(cyclohexylmethyl)pyridine-2-carboxamide (60 mg, 0.26 mmol) was treated with 4-methoxy-1-naphthalenecarbonyl chloride (71 mg, 0.32 mmol) in CH₂Cl₂ (10 mL) in the presence of DMAP (64 mg, 0.52 mmol). The crude products were purifed by MPLC on silica gel using hexane/EtOAc (4:1) to provide the title compound as a white solid (76 mg, 70%). ¹H NMR (400 MHz, CD₃OD) δ 0.90-1.04 (m, 2 H), 1.14-1.34 (m, 3 H), 1.60 (d, J=3.51 Hz, 2 H), 1.69-1.81 (m, 4 H), 3.20 (d, J=7.03 Hz, 2 H), 4.08 (s, 3 H), 7.02 (d, J=8.20 Hz, 1 H), 7.44-7.64 (m, 3 H, 7.93 (d, J=8.01 Hz, 1 H), 8.28-8.40 (m, 2 H), 8.51 (d, J=8.40 Hz, 1 H), 9.27 (dd, J=8.59, 1.37 Hz, 1 H). MS (ESI) (M+H)⁺ 418.0.

1. A compound of formula I, a diasteriomer or enantiomer of the compound, a pharmaceutically acceptable salt of the compound, diasteriomer, or enantiomer, or mixtures thereof: 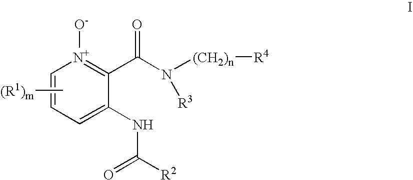
wherein:
m is 0, 1, or 2;
n is 0, 1, 2, 3, 4, or 5;
R¹ is independently selected from the group consisting of halogen, cyano, amino, nitro, C₁₋₆alkylamino, diC₁₋₆alkylamino, acetylamino, hydroxyl, C₁₋₆alkoxy, C₁₋₆alkyl, halogenated C₁₋₆alkoxy, C₁₋₆alkenyl, and halogenated C₁₋₆alkyl;
R² is C₆₋₁₀aryl or C₂₋₁₀heterocyclyl and is unsubstituted or substituted by one or more substituents selected from the group consisting of halogen, halogenated C₁₋₆alkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl;
R³ is hydrogen or C₁₋₆alkyl; and
R⁴ is selected from the group consisting of C₁₋₆alkyl, C₃₋₇cycloalkyl, C₄₋₇cycloalkenyl, C₆₋₁₀aryl, C₂₋₆heterocyclyl-amino, C₂₋₆heterocyclyloxy-amino, and C₂₋₆heterocyclyl, and is unsubstituted or substituted by one or more substituents selected from the group consisting of halogen, halogenated C₁₋₆alkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl; or
wherein the moiety 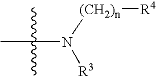
is C₂₋₁₀heterocyclyl which is unsubstituted or substituted by one or more substituents selected from the group consisting of halogen, halogen substituted C₁₋₆alkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl.

2. The compound as claimed in claim 1, wherein:
m is 0, 1, or 2;
n is 0, 1, 2, 3, or 4;
R¹ is independently selected from the group consisting of halogen, cyano, amino, nitro, acetylamino, hydroxyl, C₁₋₃alkoxy, C₁₋₃alkyl, halogenated C₁₋₃alkoxy, and halogenated C₁₋₃alkyl;
R² is C₆₋₁₀aryl or C₂₋₁₀heterocyclyl, and is unsubstituted or substituted by one or more substituents selected from the group consisting of halogen, halogenated C₁₋₃alkyl, C₁₋₃alkyl, nitro, C₁₋₃alkoxy, halogenated C₁₋₃alkoxy, hydroxy, hydroxy-C₁₋₃alkyl, amino, C₁₋₃alkoxy-C₁₋₃alkyl, C₂₋₅heterocyclyl-C₁₋₃alkyl, C₁₋₆alkoxycarbonyl, C₁₋₃alkylamino, diC₁₋₃alkyl-amino, and amino-C₁₋₃alkyl;
R³ is hydrogen or C₁₋₆ alkyl; and
R⁴ is selected from the group consisting of C₁₋₆alkyl, C₃₋₇cycloalkyl, C₂₋₆heterocyclyl-amino, C₂₋₆heterocyclyloxy-amino, and C₂₋₆ heterocyclyl and is unsubstituted or substituted by one or more substituents selected from the group consisting of halogen, halogenated C₁₋₃alkyl, C₁₋₃alkyl, nitro, C₁₋₃alkoxy, halogenated C₁₋₃alkoxy, hydroxy, hydroxy-C₁₋₃alkyl, amino, C₁₋₃alkoxy-C₁₋₃alkyl, C₁₋₆alkoxycarbonyl, C₁₋₃alkylamino, diC₁₋₃alkyl-amino, and amino-C₁₋₃alkyl; or
the moiety 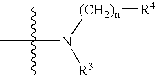
is a ring system selected from the group consisting of azepanyl, pyrrolyl, pyrrolinyl, pyrrolidinyl, imidazolyl, imidazolidinyl, pyrazolyl, pyrazolinyl, pyrazolidinyl, isoxazolidinyl, triazolyl, morpholinyl, piperidinyl, thiomorpholinyl, pyridazinyl, piperazinyl, triazinyl, and 1,4-dioxa-8-azaspiro[4.5]decan-8-yl, substituted by one or more substituents selected from the group consisting of halogen, halogenated C₁₋₃alkyl, C₁₋₃alkyl, nitro, C₁₋₃alkoxy, halogenated C₁₋₃alkoxy, hydroxy, hydroxy-C₁₋₃alkyl, amino, C₁₋₃alkoxy-C₁₋₃alkyl, C₁₋₆alkoxycarbonyl, C₁₋₃alkylamino, diC₁₋₃alkyl-amino, and amino-C₁₋₃alkyl.

3. The compound as claimed in claim 1, wherein
m is 0, 1, or 2;
n is 0, 1, 2, 3, or 4;
R¹ is independently selected from the group consisting of halogen, amino, nitro, acetylamino, hydroxyl, C₁₋₃alkoxy, C₁₋₃alkyl, halogenated C₁₋₃alkoxy, and halogenated C₁₋₃alkyl;
R² is selected from the group consisting of phenyl, naphthyl, pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4 oxadiazolyl, indolyl, indolinyl, quinolinyl, tetrahydroquinolinyl, isoquinolinyl, tetrahydro-isoquinolinyl, 1,4-benzodioxanyl, coumarin, dihydrocoumarinyl, 2,3-dihydrobenzofuranyl, 1,2-benzisoxazolyl, 1,3-benzodioxolyl, 2,3-dihydro-1,4-benzodioxinyl, 3,4-dihydro-2H-1,5-benzo-dioxepinyl, 4H-1,3-benzodioxinyl, benzofuranyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl, and is unsubstituted or substituted by one or more substituents selected from the group consisting of halogen, hydroxy, methyl, methoxy, amino, trifluoromethyl, trifluoromethoxy, methoxymethyl, 1H-1,2,3-triazolylmethyl, and 1H-pyrazolylmethyl;
R³ is hydrogen or C₁₋₆ alkyl; and
R⁴ is a ring system selected from the group consisting of 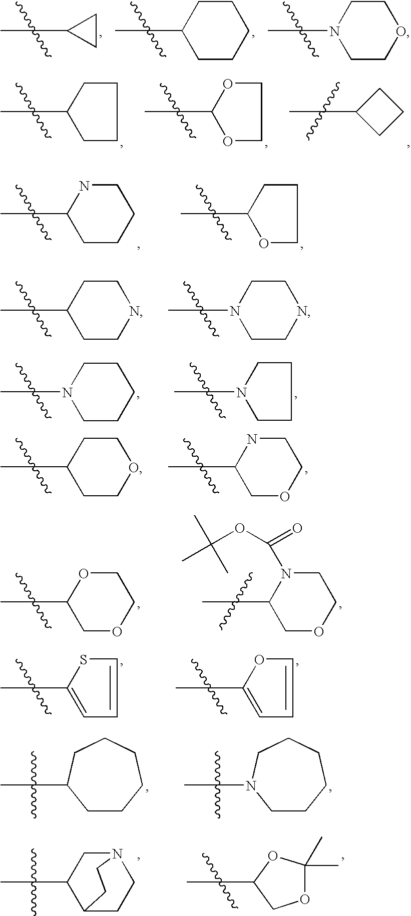
pyrrolidin-1-amino, piperidin-1-amino, O-cyclohexylhydroxyamino, O-cyclopentylhydroxy-amino, O-cyclobutylhydroxyamino, O-cyclopropylhydroxyamino, and C₁₋₃alkyl and is unsubstituted or substituted by one or more substituents selected from the group consisting of halogen, amino, aminomethyl, 2-aminoethyl, hydroxy, hydroxylmethyl, methyl, and ethyl.

4. The compound as claimed in claim 3, wherein
R² is a ring system selected from the group consisting of: 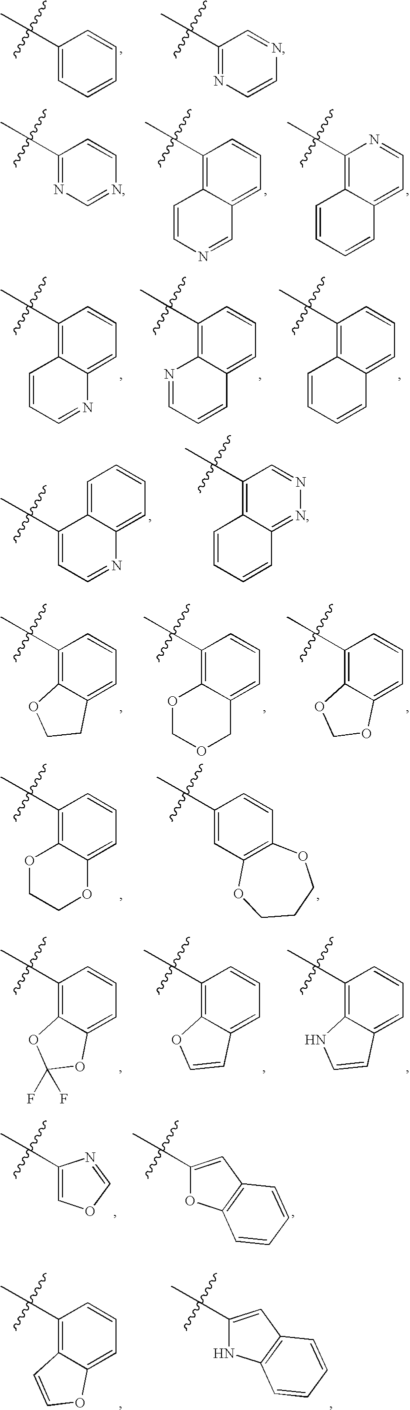 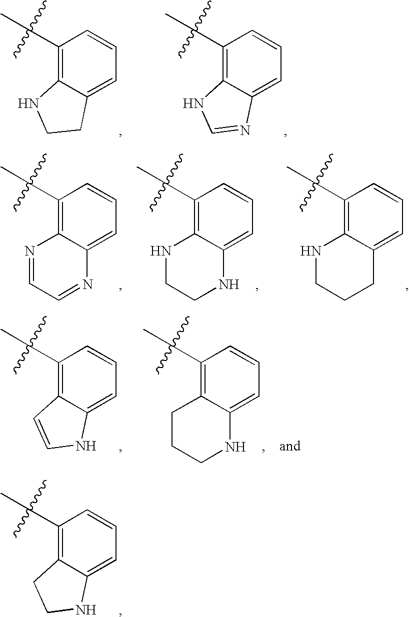
and is unsubstituted or substituted with one or more substituents selected from the group consisting of halogen, methyl, methoxy, hydroxyl, methoxymethyl, 1H- 1,2,3-triazolylmethyl, and 1H-1,2-diazolylmethyl.

5. The compound as claimed in claim 1, wherein:
m is 1;
n is 0, 1, 2, or 3;
R¹ is independently selected from the group consisting of halogen, amino, nitro, acetylamino, hydroxyl, C₁₋₃alkoxy, C₁₋₃alkyl, halogenated C₁₋₃alkoxy, and halogenated C₁₋₃alkyl;
R² is selected from the group consisting of phenyl, naphthyl, pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4 oxadiazolyl, indolyl, indolinyl, quinolinyl, tetrahydroquinolinyl, isoquinolinyl, tetrahydro-isoquinolinyl, 1,4-benzodioxanyl, coumarin, dihydrocoumarinyl, 2,3-dihydrobenzofuranyl, 1,2-benzisoxazolyl, 1,3-benzodioxolyl, 2,3-dihydro-1,4-benzodioxinyl, 3,4-dihydro-2H-1,5-benzo-dioxepinyl, 4H-1,3-benzodioxinyl, benzofuranyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl, and is unsubstituted or substituted by one or more substituents selected from the group consisting of halogen, hydroxy, methyl, methoxy, amino, trifluoromethyl, trifluoromethoxy, methoxymethyl, 1H-1,2,3-triazolylmethyl, and 1H-pyrazolylmethyl; and
the moiety 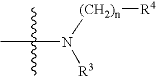
is a ring system selected from the group consisting of azetidinyl, azepanyl, isoxazolidinyl, morpholinyl, piperazinyl, piperidinyl, pyrrolidinyl, and 1,4-dioxa-8-azaspiro[4.5]decan-8-yl and is unsubstituted or substituted with one or more substituents selected from the group consisting of halogen, cyano, nitro, methyl, ethyl, hydroxy, hydroxy-methyl, hydroxy-ethyl, amino-methyl, amino-ethyl, methoxy-methyl, methoxy-phenyl, ethoxycarbonyl, tert-butoxycarbonyl, diphenyl-methyl, morpholinyl-eth-2-yl, piperidinyl-methyl and pyridinyl.

6. The compound as claimed in claim 5, wherein the moiety 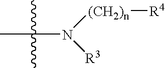
is a ring system selected from the group consisting of:

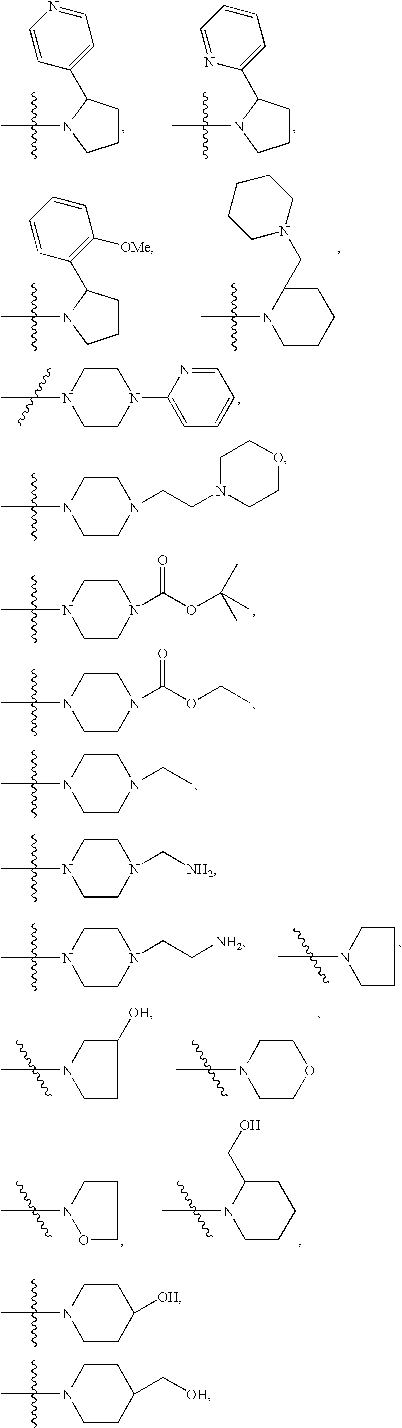

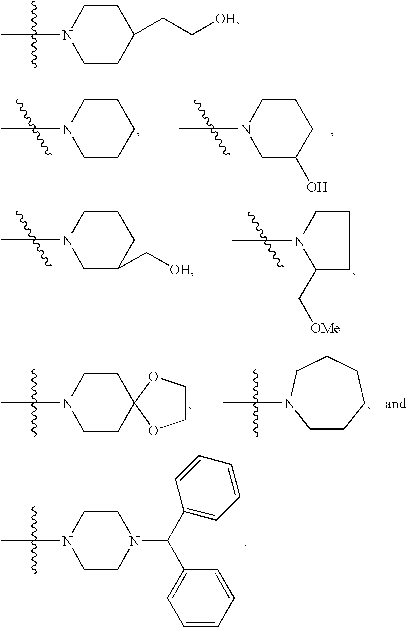

7. The compound as claimed in claim 5, wherein R² is selected from the group consisting of: 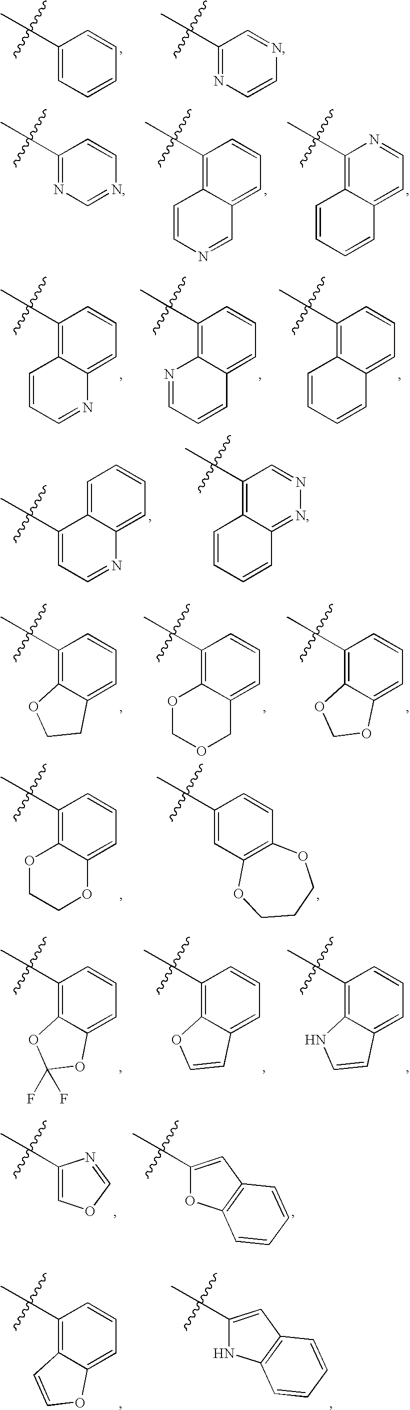 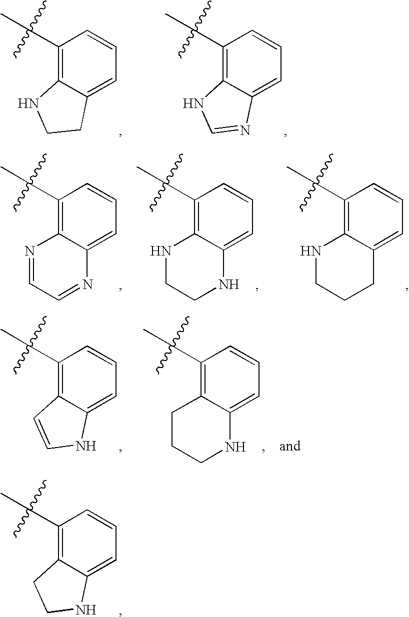
and is unsubstituted or substituted with one or more substitutents selected from the group consisting of halogen, methyl, methoxy, hydroxyl, methoxymethyl, 1H-1,2,3-triazolylmethyl, and 1H-pyrazolylmethyl.

8. The compound as claimed in claim 1, wherein R² is selected from the group consisting of 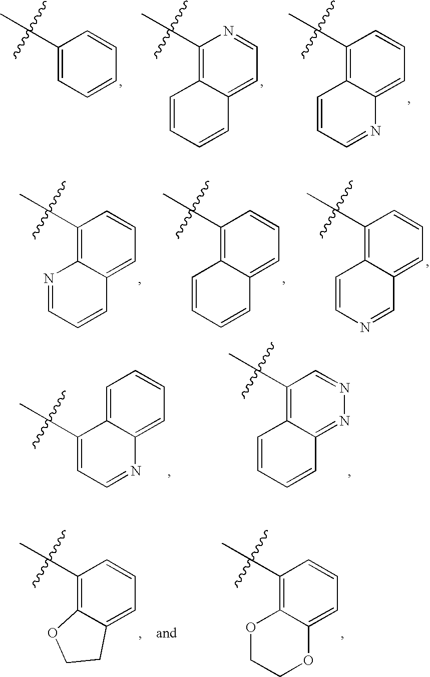
and is unsubstituted or substituted with one or more substituents selected from the group consisting of halogen, methyl, methoxy, hydroxyl, methoxymethyl, 1H-1,2,3-triazolylmethyl and 1H-pyrazolylmethyl.

9. A compound selected from the group consisting of:
N-(Cyclobutylmethyl)-3-[(1-naphthalenylcarbonyl)amino]-1-oxide-2-pyridinecarboxamide;
N-(Cyclobutylmethyl)-3-[[(4-methyl-1-naphthalenyl)carbonyl]amino]-1-oxide-2-pyridine-carboxamide;
N-(Cyclobutylmethyl)-3-[[(4-methoxy-1-naphthalenyl)carbonyl]amino]-1-oxide-2-pyridine-carboxamide;
3-[(1-Naphthalenylcarbonyl)amino]-N-[(tetrahydro-2H-pyran-4-yl)methyl]-1-oxide-2-pyridine-carboxamide;
3-[[(4-Methyl-1-naphthalenyl)carbonyl]amino]-N-[(tetrahydro-2H-pyran-4-yl)methyl]-1-oxide-2-pyridinecarboxamide;
3-[[(4-Methoxy-1-naphthalenyl)carbonyl]amino]-N-[(tetrahydro-2H-pyran-4-yl)methyl]-1-oxide-2-pyridinecarboxamide;
N-(Cyclohexylmethyl)-3-[(1-naphthalenylcarbonyl)amino]-1-oxide-2-pyridinecarboxamide;
N-(Cyclohexylmethyl)-3-[(4-methyl-1-naphthalenylcarbonyl)amino]-1-oxide-2-pyridine-carboxamide;
N-(Cyclohexylmethyl)-3-[(4-methoxy-1-naphthalenylcarbonyl)amino]-1-oxide-2-pyridine-carboxamide;
N-(Cyclohexylmethyl)-3-[(4-methoxy-1-naphthalenylcarbonyl)amino]-1-oxide-2-pyridine-carboxamide;
or a diasteriomer or enantiomer of the compound or a pharmaceutically acceptable salt of the compound, diasteriomer, or enantiomer.

10. (canceled)

11. A method for the treatment of pain, the method comprising administering an effective amount of a compound according to claim 1 to a patient in need thereof.

12-13. (canceled)

14. A method for the treatment of a medical condition selected from the group consisting of anxiety, cancer, multiple sclerosis, Parkinson's disease, Huntington's chorea, Alzheimer's disease, and cardiovascular disorders, the method comprising administering a therapeutically effective amount of the compound according to claim 1 to a patient in need thereof.

15. A pharmaceutical composition comprising a compound according to claim 1 and a pharmaceutically acceptable carrier.

16. A method for the treatment of a functional gastrointestinal disorder, the method comprising the step of administering a therapeutically effective amount of a compound according to claim 1 to a patient in need thereof.

17. A method for the treatment of irritable bowel syndrome in a patient, the method comprising the step of administering a therapeutically effective amount of a compound according to claim 1 to a patient in need thereof.

18. A method for preparing a compound of formula I, 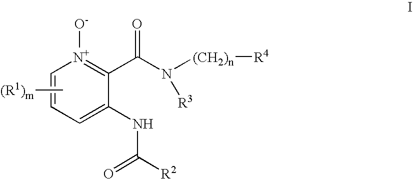
the method comprising the step of reacting a compound of formula II, 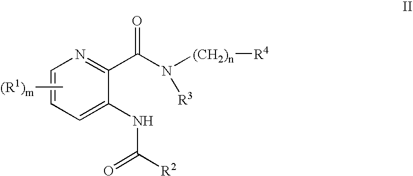
with mCPBA, in the presence of a solvents,
wherein:
m is 0, 1, or 2;
n is 0, 1, 2, 3, 4, or 5;
R¹ is independently selected from the group consisting of halogen, cyano, amino, nitro, C₁₋₆alkylamino, diC₁₋₆alkylamino, acetylamino, hydroxyl, C₁₋₆alkoxy, C₁₋₆alkyl, halogenated C₁₋₆alkoxy, C₁₋₆alkenyl, and halogenated C₁₋₆alkyl;
R² is C₆₋₁₀aryl or C₂₋₁₀heterocyclyl and is unsubstituted or substituted by one or more substituents selected from the group consisting of halogen, halogenated C₁₋₆alkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl;
R³ is hydrogen or C₁₋₆alkyl; and
R⁴ is selected from the group consisting of C₁₋₆alkyl, C₃₋₇cycloalkyl, C₄₋₇cycloalkenyl, C₆₋₁₀aryl, C₂₋₆heterocyclyl-amino, C₂₋₆heterocyclyloxy-amino, and C₂₋₆heterocyclyl, and is unsubstituted or substituted by one or more substituents selected from the group consisting of halogen, halogenated C₁₋₆alkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₂₋₅heterocyclyl-C₁₋₃alkyl C₃₋₆cycloalkyl, C₂₋₆heteroaryl, heteroaryl-C₁₋₆alkyl, C₆₋₁₀aryl, and C₆₋₁₀aryl-C₁₋₆alkyl; or
wherein the moiety 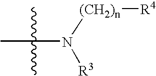
is C₂₋₁₀heterocyclyl, and is unsubstituted or substituted by one or more substituents selected from the group consisting of halogen, halogen substituted C₁₋₆alkyl, C₁₋₆alkyl, cyano, nitro, C₁₋₆alkoxy, halogenated C₁₋₆alkoxy, hydroxy, hydroxy-C₁₋₆alkyl, amino, C₁₋₆alkoxy-C₁₋₆alkyl, C₁₋₆alkylcarbonyl, C₁₋₆alkoxycarbonyl, C₁₋₆alkylamino, diC₁₋₆alkyl-amino, amino-C₁₋₆alkyl, C₃₋₆cycloalkyl, C₂₋₆heteroaryl, heteroaryl-C₁₋₆alkyl, C₆₋₁₀aryl, and C₆₋₁₀aryl-C₁₋₆alkyl.

19. The method according to claim 18, wherein the solvent is CH₂Cl₂.

20. The compound as claimed in claim 6, wherein R² is selected from the group consisting of: 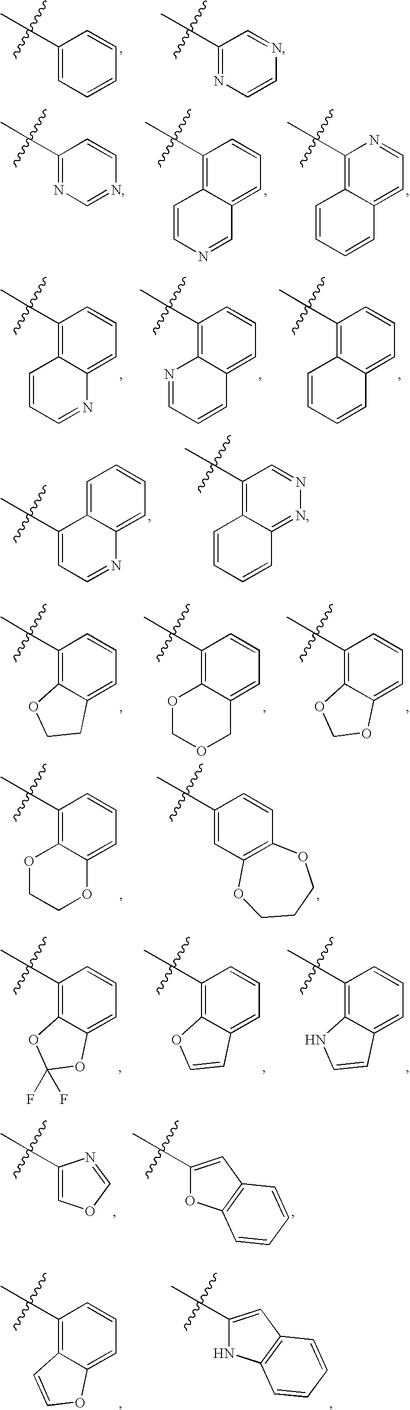 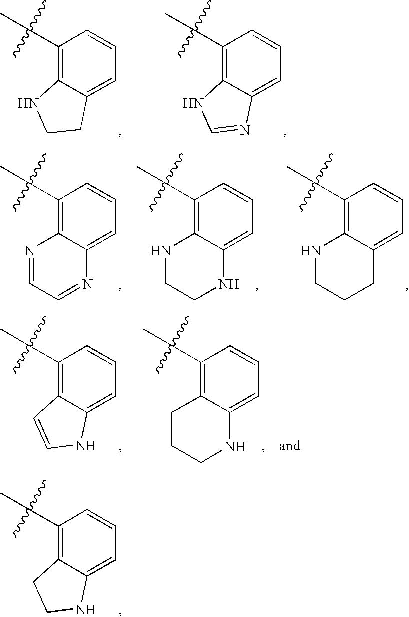
and is unsubstituted or substituted with one or more substituents selected from the group consisting of halogen, methyl, methoxy, hydroxyl, methoxymethyl, 1H-1,2,3-triazolylmethyl, and 1H-pyrazolylmethyl.

21. A method for the inhibition of transient lower esophageal sphincter relaxations, the method comprising administering a therapeutically effective amount of a compound according to claim 1 to a patient in need thereof.

22. A method for the treatment of gastroesophageal reflux disorder (GERD), the method comprising administering a therapeutically effective amount of a compound according to claim 1 to a patient in need thereof.

23. A method for the treatment of reflux, the method comprising administering a therapeutically effective amount of a compound according to claim 1 to a patient in need thereof.
